(19) United States
(12) Patent Application Publication
(10) Pub. No.: US 2020/0408771 A1
(43) Pub. Date: Dec. 31, 2020

(54) CD153 AND/OR CD30 IN INFECTION

(71) Applicant: The United States of America,as represented by the Secretary,Department of Health and Human Services, Bethesda, MD (US)

(72) Inventors: Daniel L. Barber, Rockville, MD (US); Michelle A. Sallin, Baltimore, MD (US); Keith D. Kauffman, Bowie, MD (US); Taylor Foreman, Bethesda, MD (US)

(73) Assignee: The United States of America,as represented by the Secretary,Department of Health and Human Services, Bethesda, MD (US)

(21) Appl. No.: 16/971,144

(22) PCT Filed: Feb. 22, 2019
(86) PCT No.: PCT/US2019/019164

§ 371 Date: Aug. 19, 2020

Related U.S. Application Data
(60) Provisional application No. 62/633,816, filed on Feb. 22, 2018.

(51) Int. Cl. G01N 33/68; A61K 35/17; G01N 33/569

(52) U.S. Cl. CPC G01N 33/68; A61K 35/17; G01N 33/5695; G01N 2800/56; G01N 2333/70575; G01N 2333/70578; G01N 2800/26

(57) ABSTRACT

In an embodiment, the invention provides a method of diagnosing infection in a subject. In an embodiment, the invention provides a method of determining the latency of infection in a subject. In an embodiment, the invention provides a method of determining the effectiveness of a vaccine against infection in a subject. In an embodiment, the invention provides a method of determining the severity of infection in a subject. In an embodiment, the invention provides a method of preventing or treating infection in a subject.

### CROSS-REFERENCE TO RELATED APPLICATION

This patent application claims the benefit of U.S. Provisional Patent Application No. 62/633,816, filed Feb. 22, 2018, which is incorporated by reference herein in its entirety.

### STATEMENT REGARDING FEDERALLY SPONSORED RESEARCH OR DEVELOPMENT

This invention was made with Government support under project number 1 ZIA AI001171 by the National Institutes of Health, National Institute of Allergy and Infectious Diseases. The Government has certain rights in the invention.

### BACKGROUND OF THE INVENTION

There is a continued need for additional methods for diagnosing and treating infection.

### BRIEF SUMMARY OF THE INVENTION

In an embodiment, the invention provides a method of diagnosing infection in a subject, the method comprising obtaining a biological sample from the subject; detecting the expression level of CD153 or CD30 in the sample; and diagnosing the subject with infection when the expression level of CD153 or CD30 in the sample is detected to be higher compared to the expression level of CD153 or CD30 in a subject without infection.

In an embodiment, the invention provides a method of determining the latency of infection in a subject, the method comprising obtaining a biological sample from the subject; detecting the expression level of CD153 or CD30 in the sample; and determining the infection of the subject to be latent when the expression level of CD153 or CD30 in the sample is detected to be higher compared to the expression level of CD153 or CD30 in a subject with active infection.

In an embodiment, the invention provides a method of determining the effectiveness of a vaccine against infection in a subject, the method comprising administering a vaccine to the subject; obtaining a biological sample from the subject; detecting the expression level of CD153 or CD30 in the sample; and determining the vaccine to be effective when the expression level of CD153 or CD30 in the sample is detected to be higher compared to the expression level of CD153 or CD30 in a subject with active infection.

In an embodiment, the invention provides a method of determining the severity of infection in a subject, the method comprising obtaining a biological sample from the subject; detecting the expression level of CD153 or CD30 in the sample; and determining the infection to be less severe when the expression level of CD153 or CD30 in the sample is detected to be higher compared to the expression level of CD153 or CD30 in a subject with active infection.

In an embodiment, the invention provides a method of preventing or treating infection in a subject, the method comprising administering to the subject an effective amount of a substance that upregulates CD153 or activates CD30 in CD4 T cells.

In an embodiment, the invention provides a method of preventing or treating infection in a subject, the method comprising administering to the subject an effective amount of CD4 T cells induced to upregulate CD153.

Additional embodiments are as described herein.

### BRIEF DESCRIPTION OF THE DRAWINGS

FIG. 1 shows a sorting strategy for gene expression analysis of CD4 T cells from the lungs of Mtb infected mice. Lung lymphocytes were isolated on day 28 post infection from Foxp3⁻GFP mice. Live CD4⁺Foxp3⁻CD44^{low} naïve cells were sorted into CD45iv⁺ (iv⁺) or CD45iv^{dim} (CD45iv⁻ or iv⁻) populations. Live CD4+Foxp3⁻CD44^{hi} effectors were sorted into four populations: KLRG1⁻CD45iv^{dim}, KLRG1⁻CD45iv⁺, KLRG1⁺CD45iv^{dim}, and KLRG1⁺CD45iv⁺ cells.

FIG. 2A is a plot showing a comparison of gene expression in lung effector CD44^{hi}KLRG1⁻CD45iv⁻ relative to CD44^{hi}KLRG1⁺CD45iv⁺ and naïve CD441^{low}CD45iv⁻ cells on day 28 post infection (shown are probes that are significantly different in both comparisons). The interior box indicates genes that are up-regulated in the CD44^{hi}KLRG1⁻CD45iv⁻ cells. The probes designated by arrows represent members of the TNFSF/TNFRSF families that were significant and up by >0.3 log 2 in both comparisons.

FIG. 2B presents representative FACS plots of the expression of CD153 on lung CD4 and CD8 T cells after in vitro stimulation with either ESAT-6₁₋₂₀ or TB10.4₄₋₁₁.

FIG. 2C is a line graph of the quantification of the expression of CD153 and CX3CR1 on lung CD4 T cells after in vitro stimulation with ESAT-6₁₋₂₀. Vertical bars represent the standard error in three to five samples per time point.

FIG. 2D presents survival curves of WT and Tnfsf8^{-/-} mice infected with ˜100 colony forming units (CFU or c.f.u.) of Mtb. Each survival curve is representative of 4 independent experiments.

FIG. 2E is a dot plot of lung and spleen CFU of WT and Tnfsf8^{-/-} mice. Data from three experiments done on days 82 and 89 post-infection are shown. Horizontal bars represent the mean values of three independent experiments pooled. Statistical significance determined by d, Mantel-Cox and e, f, and g two-tailed t tests: **p≤0.01, ***p≤0.001, ****p≤0.0001

FIG. 3A presents FACS histograms of the expression of GITR on naïve and effector CD4 T cells, iv⁺KLRG1⁺, iv⁺KLRG1⁺, and iv⁺KLRG⁺ in the lung.

FIG. 3B presents survival curves of WT and GITRL^{-/-} mice infected with ˜100 CFU Mtb. Statistical significance determined by the Mantel-Cox test.

FIG. 3C presents FACS histograms of the expression of OX40 on naïve and effector CD4 T cells, iv⁻KLRG1⁻, iv⁻KLRG1⁺, and iv⁺KLRG1⁺ in the lung.

FIG. 3D presents survival curves of WT and OX40^{-/-} mice infected with ˜100 CFU Mtb. Statistical significance determined by the Mantel-Cox test.

FIG. 3E presents FACS histograms of the expression of RANKL on naïve and effector CD4 T cells, iv⁻KLRG1⁻, iv⁻KLRG1⁺, and iv⁺KLRG1⁺ in the lung.

FIG. 3F presents survival curves of RANKL-fl and RANKL-fl x CD4-Cre mice infected with ˜100 CFU Mtb. Statistical significance determined by the Mantel-Cox test.

FIG. 4A presents representative FACS plots of I-A^{b}ESAT-6₄₋₁₇ tetramer and intravascular staining of lung effector CD4⁺ T cells from WT and Tnfsf8^{-/-} mice.

FIG. 4B presents dot plots showing the quantification of I-A^{b}ESAT-6₄₋₁₇ tetramer⁺CD4 T cells of FIG. 4A. Horizontal bars represent the mean values of two independent experiments pooled. Horizontal bars represent the mean values of two independent experiments pooled.

FIG. 4C presents dot plots showing the quantification of the percentage of I-A^{b}ESAT-6₄₋₁₇ tetramer⁺CD4 T cells of FIG. 4A that are intravascular stain negative. Horizontal bars represent the mean values of two independent experiments pooled.

FIG. 4D presents representative FACS plots of the expression of IFNγ and CD153 and quantification of the expression of IFNγ by WT and Tnfsf8^{-/-} Mtb-specific CD4 T cells from the lung after in vitro stimulation with ESAT-6₁₋₂₀ peptide. Lines pair the unstimulated and stimulated wells for each mouse. Data are pooled from two independent experiments.

FIG. 4E presents representative FACS plots of the expression IFNγ vs TNF and CD153 vs CD154 from WT and IFNγ^{-/-} Mtb-specific CD4 T cells from the lung after in vitro stimulation with ESAT-6₁₋₂₀ peptide. Quantification of the frequency of CD153 expression in WT and IFNγ^{-/-} Mtb-specific CD4 T cells. Horizontal bars represent the mean of two independent experiments pooled.

FIG. 4F presents representative FACS plots of CD154 vs. TNF expression by WT, T-bet^{+/-}, and T-bet^{-/-} Mtb-specific CD4 T cells after in vitro stimulation with ESAT-6₁₋₂₀ peptide. Representative FACS plots of CD153 vs IFNγ of the CD154⁺ Mtb-specific CD4 T cells from the lungs WT, T-bet^{+/-}, and T-bet^{-/-} mice. Quantification of the frequency of CD153 or IFNγ expression in CD154⁺ Mtb-specific CD4 T cells from the lungs WT, T-bet^{+/-}, and T-bet^{-/-} mice.

FIG. 4G presents survival curves of T-cell deficient mice receiving either naïve CD153^{-/-}, IFNγ^{-/-}, WT CD4 T donor cells prior to Mtb infection.

FIG. 5A presents example fluorescence-activated cell sorting (FACS) plots of either CD4 or CD8 T cells from either bronchoalveolar lavage (BAL) or peripheral blood (PBMCs) following restimulation with MTB300 peptide pool, depicting TNF and CD153 staining.

FIG. 5B presents a line graph showing quantification of the percent of TNF⁺ CD4 T cells following restimulation with MTB300 peptide pool which co-express CD153 at various time points following infection with Mtb Erdman-mCherry. Error bars represent range of values for each tissue and time point. Data is representative of two separate experiments.

FIG. 5C presents a dot plot of the quantification of the percentage of TNF⁺IFNγ⁺ CD4 T cells following stimulation with either ESAT-6/CFP-10 peptide pools or MTB300 pools that co-express CD153 from various tissues at necropsy. Animals from experiment #1 are represented by open symbols and were infected with ˜8 CFU of Mtb Erdman and were restimulated with ESAT-6/CFP-10 peptide pools. Animals from experiment #2 are represented by filled symbols and were infected with 50-80 CFU of Mtb Erdman-mCherry and were restimulated with MTB300 peptide pool. Statistical significance for the pairwise comparison of the geometric mean value of each tissue is listed in Table 1.

FIG. 5D presents a dot plot showing correlation between the percentage of peptide-specific CD4 T cells in granulomas which co-express CD153 following restimulation and the bacterial burden of each individual granuloma. Data is taken from both experiments.

FIG. 6A presents a representative example of the expression of CD153 in MTB300-specific CD4 T cells in active or latent Mtb infection.

FIG. 6B presents a representative example of the expression of HLA-DR in MTB300-specific CD4 T cells in active or latent Mtb infection.

FIG. 6C presents a dot plot showing comparison of the frequency of CD153 (FIG. 6A) and HLA-DR (FIG. 6B) expression in MTB300-specific CD4 T cells in persons with active TB (aTB, n=8) or latent Mtb infection (LTBI, n=8). Medians and interquartile ranges are shown. Statistical comparisons were performed using a Mann-Whitney t-test.

FIG. 6D presents a representative example of CD153 expression in MTB300-specific TNFa⁺CD8 T cells in one individual with active TB infection.

FIG. 6E is a dot plot showing polyfunctional potential of MTB300-specific CD4 T cells in aTB (n=8; dots on left for each bin) and LTBI (n=8; dots on right for each bin). Medians and interquartile ranges are depicted. Statistical comparisons were performed using a Wilcoxon rank-sum test.

FIG. 7A presents example fluorescence-activated cell sorting (FACS) plots of CD4 T cells. Cells from a C57BL/6-T-bet-ZsGreen[Tg] mouse isolated from lungs at day 42 post Mtb infection, stimulated with ESAT-6₁₋₂₀ peptide, and stained. CD4 T cells which were TNF⁺IFNγ⁺ were also analyzed for CD30 expression.

FIG. 7B presents example fluorescence-activated cell sorting (FACS) plots of CD4 T cells. Analysis of CD4 T cells from BAL fluid of rhesus macaques on day 42 post Mtb infection for CD30 expression following stimulation with MTB300 peptide pool.

FIG. 7C presents example fluorescence-activated cell sorting (FACS) plots of CD4 T cells. Analysis of peripheral blood CD4 T cells following stimulation with MTB300 peptide in a clinically latent human individual for CD30 expression.

FIG. 8A shows FACS and line graphs showing comparison of the expression of KLRG1 between MTB300-specific CD153⁺ TNF⁺CD4 T cells and MTB300-specific CD153⁻ TNF⁺CD4 T cells in persons with active TB (aTB, n=8) or latent TB infection (LTBI, n=8). Medians and interquartile ranges. Statistical comparisons were performed using a Mann-Whitney t-test.

FIG. 8B shows FACS and line graphs showing comparison of the expression of HLA-DR between MTB300-specific CD153⁺ TNF⁺CD4 T cells and MTB300-specific CD153-TNF⁺CD4 T cells in persons with active TB (aTB, n=8) or latent TB infection (LTBI, n=8). Medians and interquartile ranges. Statistical comparisons were performed using a Mann-Whitney t-test.

FIG. 9 is a line graph showing survival of WT, CD30 and CD153 KO mice after infection with Mtb by aerosol.

FIG. 10: CD30 is primarily expressed on macrophages and CD153 on CD4 T cells in the lungs of Mtb infected mice. Various immune cell subsets were FACS purified from the lungs of mice infected with Mtb. SiglecF+alveolar macrophages, CD11b+lung parenchymal macrophages, lung parenchymal neutrophils, and lung parenchymal CD4 T cells were obtained, and CD30 and CD153 mRNA were measured by quantitative PCR.

FIG. 11A presents line graphs showing mice infected with Leishmania major in the ear skin, with the lesion size measured over time.

FIG. 11B is a dot plot showing mice infected with Leishmania major in the ear skin, with parasite loads quantified in the lesions 22 weeks post-infection.

FIG. 12A: Mice were infected with Leishmania major. T cell responses in the ear lesions were quantified 22 weeks post-infection. Cells from the ear were restimulated with PMA/Ionomycin as a positive control or with soluble leishmania antigen (SLA) to detect parasite specific T cells. A parasite-specific T cell response can be detected as IFNγ/TNFα positive cells after restimulation with SLA.

FIG. 12B: Mice were infected with Leishmania major. T cell responses in the ear lesions were quantified 22 weeks post-infection. WT L. major specific CD4 T cells express very high levels of CD153.

FIG. 13A shows worm burdens in the bronchoalveolar lavage fluid after mice were infected with Ascaris eggs and analyzed on day 8 post infection.

FIG. 13B shows that both Th1 and Th2 cells express CD153 after mice were infected with Ascaris eggs and analyzed on day 8 post infection. CD4 T cells were restimulated with PMA/Ionomycin to detect IFNγ-producing Th1 cells and IL-13 producing Th2 cells. Th2 cells CD153 express higher levels compared to Th1 cells during roundworm infection.

### DETAILED DESCRIPTION OF THE INVENTION

In an embodiment, the invention provides a method of diagnosing infection in a subject, the method comprising obtaining a biological sample from the subject; detecting the expression level of CD153 or CD30 in the sample; and diagnosing the subject with infection when the expression level of CD153 or CD30 in the sample is detected to be higher compared to the expression level of CD153 or CD30 in a subject without infection. In an embodiment, the expression level of CD153 is detected. In an embodiment, the expression level of CD30 is detected. In an embodiment, the higher CD153 expression level is due to expression in CD4 T cells. In an embodiment, the biological sample is peripheral blood. In an embodiment, the biological sample is bronchoalveolar lavage fluid. In an embodiment, the infection is Mycobacterium tuberculosis infection (Mtb). In an embodiment, the Mtb infection is a pulmonary Mtb infection. In an embodiment, the infection is a parasitic infection. In an embodiment, the parasite is Leishmania major or Ascaris roundworm.

In an embodiment, the invention provides a method of determining the latency of infection in a subject, the method comprising obtaining a biological sample from the subject; detecting the expression level of CD153 or CD30 in the sample; and determining the infection of the subject to be latent when the expression level of CD153 or CD30 in the sample is detected to be higher compared to the expression level of CD153 or CD30 in a subject with active infection. In an embodiment, the expression level of CD153 is detected. In an embodiment, the expression level of CD30 is detected. In an embodiment, the higher CD153 expression level is due to expression in CD4 T cells. In an embodiment, the biological sample is peripheral blood. In an embodiment, the biological sample is bronchoalveolar lavage fluid. In an embodiment, the infection is Mycobacterium tuberculosis infection (Mtb). In an embodiment, the sample is contacted with a Mtb antigen. In an embodiment, the Mtb infection is a pulmonary Mtb infection. In an embodiment, the infection is a parasitic infection. In an embodiment, the parasite is Leishmania major or Ascaris roundworm.

In an embodiment, the invention provides a method of determining the effectiveness of a vaccine against infection in a subject, the method comprising administering a vaccine to the subject; obtaining a biological sample from the subject; detecting the expression level of CD153 or CD30 in the sample; and determining the vaccine to be effective when the expression level of CD153 or CD30 in the sample is detected to be higher compared to the expression level of CD153 or CD30 in a subject with active infection. In an embodiment, the expression level of CD153 is detected. In an embodiment, the expression level of CD30 is detected. In an embodiment, the higher CD153 expression level is due to expression in CD4 T cells. In an embodiment, the biological sample is peripheral blood. In an embodiment, the biological sample is bronchoalveolar lavage fluid. In an embodiment, the infection is Mycobacterium tuberculosis infection (Mtb). In an embodiment, the Mtb infection is a pulmonary Mtb infection. In an embodiment, the infection is a parasitic infection. In an embodiment, the parasite is Leishmania major or Ascaris roundworm.

In an embodiment, the invention provides a method of determining the severity of infection in a subject, the method comprising obtaining a biological sample from the subject; detecting the expression level of CD153 or CD30 in the sample; and determining the infection to be less severe when the expression level of CD153 or CD30 in the sample is detected to be higher compared to the expression level of CD153 or CD30 in a subject with active infection. In an embodiment, the expression level of CD153 is detected. In an embodiment, the expression level of CD30 is detected. In an embodiment, the higher CD153 expression level is due to expression in CD4 T cells. In an embodiment, the biological sample is peripheral blood. In an embodiment, the biological sample is bronchoalveolar lavage fluid. In an embodiment, the infection is Mycobacterium tuberculosis infection (Mtb). In an embodiment, the Mtb infection is a pulmonary Mtb infection. In an embodiment, the infection is a parasitic infection. In an embodiment, the parasite is Leishmania major or Ascaris roundworm.

The term “detect” and “detecting” as used herein with respect to the expression of CD153 or CD30 means to determine the presence or absence of detectable expression. Detection encompasses, but is not limited to, measuring (or quantifying) the expression level of CD153 or CD30 by any suitable method. In one embodiment, the method involves measuring the expression of CD153 or CD30 in such a way as to facilitate the comparison of expression levels between samples.

Higher expression of CD153 or CD30 can be detected by comparing the expression of CD153 or CD30 in a subject with a control (e.g., a positive or negative control). A control can be provided, for example, by measuring the expression of CD153 i or CD30 n a tissue or subject known to be negative for infection (negative control), or known to be positive for infection (positive control). The control also can be provided by a previously determined standard prepared by any suitable method (e.g., an expression profile of CD153 or CD30 generated from a population of subjects known to be positive or negative for infection). Of course, the expression level used to provide a control should be generated with respect to a subject and/or tissue of the same type as the subject and/or tissue under examination (e.g., human). When comparing the expression of CD153 or CD30 to a negative control, higher expression can be defined as any level of expression greater than the level of expression of the control (e.g., 1.5-fold, 2-fold, 5-fold, 10-fold, 20-fold, 50-fold, 100-fold or even greater expression as compared to the negative control).

Higher expression of CD153 or CD30 in a subject typically will be determined by analyzing CD153 or CD30 expression in a biological sample from the subject. The sample, as referred to herein, can be any suitable sample. Suitable samples include samples from a subject or host. The sample can be a liquid or fluid sample, such as a sample of body fluid (e.g., blood, plasma, interstitial fluid, serum, urine, synovial fluid, etc.), or a solid sample, such as a tissue sample. Typically, the method will be used with a sample of fluid or tissue from an area of the subject believed or suspected of being affected by the Mt infection (e.g., cells, tissue, or fluid of the colon or from a joint, such as cartilage, etc.). The tissue sample can be used whole or can be processed (e.g., cultured, extracted, homogenized, etc.) according to routine procedures prior to analysis.

The methods of the invention find utility as used with any subject, including a human, non-human primate, rat, mouse, cow, horse, pig, sheep, goat, dog, cat, etc. The subject of testing can be suspected of having infection, diagnosed with such an infection, of an unknown status with respect to the infection, or a control subject that is confirmed not to have infection.

The expression of CD153 or CD30 can be detected or measured by any suitable method. For example, expression of CD153 or CD30 can be detected on the basis of mRNA or protein levels. Suitable methods of detecting or measuring mRNA include, for example, Northern Blotting, reverse-transcription PCR (RT-PCR), and real-time RT-PCR. Such methods are described in Sambrook and Russell, Molecular Cloning: A Laboratory Manual, 3rd Ed., Cold Spring Harbor Laboratory Press, Cold Spring Harbor, N.Y., 2001. In real-time PCR, which is described in Bustin, J. Mol. Endocrinology 25: 169-193 (2000), PCRs are carried out in the presence of a labeled (e.g., fluorogenic) oligonucleotide probe that hybridizes to the amplicons. The probes can be double-labeled, for example, with a reporter fluorochrome and a quencher fluorochrome. When the probe anneals to the complementary sequence of the amplicon during PCR, the Taq polymerase, which possesses 5′ nuclease activity, cleaves the probe such that the quencher fluorochrome is displaced from the reporter fluorochrome, thereby allowing the latter to emit fluorescence. The resulting increase in emission, which is directly proportional to the level of amplicons, is monitored by a spectrophotometer. The cycle of amplification at which a particular level of fluorescence is detected by the spectrophotometer is called the threshold cycle, C_{T}. It is this value that is used to compare levels of amplicons. Probes suitable for detecting CD153 or CD30 mRNA levels are commercially available and/or can be prepared by routine methods, such as methods discussed elsewhere herein.

Suitable methods of detecting protein levels in a sample include flow cytometry, immunohistochemistry, immunocytochemistry, immunofluorescence, Western Blotting, radio-immunoassay, and Enzyme-Linked Immunosorbent Assay (ELISA). Such methods are described in Nakamura et al., Handbook of Experimental Immunology, 4^{th} ed., Vol. 1, Chapter 27, Blackwell Scientific Publ., Oxford, 1987. When detecting proteins in a sample using an immunoassay, the sample is typically contacted with antibodies or antibody fragments (e.g., F(ab)₂′ fragments, single chain antibody variable region fragment (scFv) chains, and the like) that specifically bind the target protein (e.g., the CD153 or CD30 protein). Antibodies and other polypeptides suitable for detecting CD153 or CD30 in conjunction with immunoassays are commercially available and/or can be prepared by routine methods, such as methods discussed elsewhere herein (e.g., Harlow and Lane, Using Antibodies: A Laboratory Manual, Cold Spring Harbor Laboratory Press, Cold Spring Harbor, N.Y., 1998).

The immune complexes formed upon incubating the sample with the antibody are subsequently detected by any suitable method. In general, the detection of immune complexes is well-known in the art and can be achieved through the application of numerous approaches. These methods are generally based upon the detection of a label or marker, such as any radioactive, fluorescent, biological or enzymatic tags or labels of standard use in the art. U.S. patents concerning the use of such labels include U.S. Pat. Nos. 3,817,837, 3,850,752, 3,939,350, 3,996,345, 4,277,437, 4,275,149 and 4,366,241.

For example, the antibody used to form the immune complexes can, itself, be linked to a detectable label, thereby allowing the presence of or the amount of the primary immune complexes to be determined. Alternatively, the first added component that becomes bound within the primary immune complexes can be detected by means of a second binding ligand that has binding affinity for the first antibody. In these cases, the second binding ligand is, itself, often an antibody, which can be termed a “secondary” antibody. The primary immune complexes are contacted with the labeled, secondary binding ligand, or antibody, under conditions effective and for a period of time sufficient to allow the formation of secondary immune complexes. The secondary immune complexes are then washed to remove any non-specifically bound labeled secondary antibodies or ligands, and the remaining label in the secondary immune complexes is then detected.

Other methods include the detection of primary immune complexes by a two-step approach. A second binding ligand, such as an antibody, that has binding affinity for the first antibody can be used to form secondary immune complexes, as described above. After washing, the secondary immune complexes can be contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. A number of other assays are contemplated; however, the invention is not limited as to which method is used.

The level of CD153 or CD30 present in a sample can be normalized to the level of a protein or other substance present in the sample. In some embodiments, the level of CD153 or CD30 present in a sample is normalized to the level of a protein encoded by a “housekeeping gene” which is expressed in the sample. The term “housekeeping gene” is well-known in the art as referring to a gene expressed at a relatively constant level during physiological and pathophysiological conditions. A protein encoded by any housekeeping gene can be used to normalize the level of CD153 or CD30 present in a sample. Non-limiting examples of housekeeping genes include GAPDH and beta-actin. In some embodiments, the protein used to normalize the level of CD153 or CD30 is encoded by a gene which is expressed in a tissue-specific, e.g., organ-specific, manner. Tissue-specific genes and their protein products are well-known to those of skill in the art. In other embodiments, the substance used for normalization represents a set of related molecules, for example, total protein in the sample. In other embodiments, the substance is not a protein but another component present in a sample, such as a nucleic acid, lipid, carbohydrate, or small organic or non-organic molecule.

Any suitable method known in the art can be used to determine the level of a protein used to normalize the level of CD153 or CD30 in a sample. In some embodiments, the method for determining the level of a normalization protein in a sample is the same as the method for determining the level of CD153 or CD30 in the sample, except, e.g., in ELISA an antibody specific for the normalization protein is substituted for an anti-CD153 or anti-CD30 antibody.

The method of detecting infection can be used for any purpose. For example, the method of detecting infection can be used to screen for disease or assist in making a clinical diagnosis. Alternatively, or in addition, the method of detecting infection can be used to distinguish between affected and unaffected tissues in a given area of the body (e.g., adjacent tissues), as might be useful in delineating the border of tissue to be surgically removed. The method of detecting infection also can be used to monitoring the progression or regression of such a condition or disease in a subject. In this respect, the method of detecting infection can further comprise (a) measuring the CD153 or CD30 expression level in a first sample obtained from the subject at a first point in time, (b) measuring the CD153 e or CD30 xpression level in a second sample obtained from the subject at a second point in time, and (c) comparing the CD153 or CD30 expression levels of the first and second samples. Comparison of the expression of CD153 or CD30 can be performed by directly comparing the CD153 e or CD30 xpression level of the first sample with that of the second sample. Alternatively, or in addition, the CD153 or CD30 expression levels of the first and second samples can be indirectly compared to each other by comparing the expression level of each sample to a control. A control can be provided as previously described herein. A difference in the expression level as between the first and second samples indicates a change in the status of the disease, wherein increasing expression levels between an earlier point in time and a later point in time suggests progression of the disease and a decrease in the expression levels between an earlier point in time and a later point in time suggests a regression of the disease. No difference in the expression levels suggests stasis of the condition. Such methods can be useful not only for detecting infection, but also for prognosticating the course of the disease or condition, establishing toxic limits of a drug, developing dosing regimens, or monitoring the effectiveness of a particular treatment for infection.

The method of detecting infection can further comprise, in addition to detecting higher expression of CD153 or CD30, detecting or measuring the expression of other biomarkers associated with infection. Non-limiting examples of biomarkers include HLA-DR, CD38, and Ki67 expression by Mtb-specific T cells in Mtb.

In an embodiment, the invention provides a method of preventing or treating infection in a subject, the method comprising administering to the subject an effective amount of a substance that upregulates CD153 or activates CD30 in CD4 T cells. In an embodiment, the infection is Mycobacterium tuberculosis infection (Mtb). In an embodiment, the Mtb infection is a pulmonary Mtb infection. In an embodiment, the infection is a parasitic infection. In an embodiment, the parasite is Leishmania major or Ascaris roundworm. In an embodiment, the substance upregulates CD153. In an embodiment, the substance activates CD30.

In an embodiment, the invention provides a method of preventing or treating infection in a subject, the method comprising administering to the subject an effective amount of CD4 T cells induced to upregulate CD153. In an embodiment, the infection is Mycobacterium tuberculosis infection (Mtb). In an embodiment, the Mtb infection is a pulmonary Mtb infection. In an embodiment, the infection is a parasitic infection. In an embodiment, the parasite is Leishmania major or Ascaris roundworm. In an embodiment, the CD4 T cells are taken from the subject and administered using adoptive cell transfer.

In an embodiment, the invention provides a method of treating infection in a subject, the method comprising receiving an identification of the subject as having a higher expression level of CD153 or CD30 when compared to the expression level of CD153 or CD30 in a subject without infection, and administering to the subject an effective amount of a substance that treats the infection. Such a substance may upregulate CD153 or activate CD30 in CD4 T cells. In an embodiment, the infection is Mycobacterium tuberculosis infection (Mtb). In an embodiment, the Mtb infection is a pulmonary Mtb infection. In an embodiment, the infection is a parasitic infection. In an embodiment, the parasite is Leishmania major or Ascaris roundworm.

In any embodiment of the invention, the infection can be treated. Treatment of latent infection may require just a single therapeutic. Treatment of active infection often requires several therapeutics at once. Common therapeutics for Mtb include, e.g., isoniazid, rifampin (Rifadin, Rimactane), ethambutol (Myambutol), rifapentine, and pyrazinamide. For drug-resistant TB, fluoroquinolones can be used in combination with injectable therapeutics, such as amikacin, kanamycin, and capreomycin, and other second-line drugs include cycloserine, azithromycin, clarithromycin, moxifloxacin, and levofloxacin. Common therapeutics for L. major include, e.g., sodium stibogluconate, liposomal amphotericin B, miltefosine, amphotericin B deoxycholate, pentamidine isethionate, ketoconazole, itraconazole, fluconazole, paromomycin. Common therapeutics for Ascaris roundworm infection include, e.g., albendazole, ivermectin, and mebendazole.

Treatment can be linked to the diagnosis of infection, determination of latency of infection, and/or determination of severity for the infection. For example, upon diagnosis of infection, appropriate and effective treatment can be initiated. The treatment may be altered upon determination of latency of infection or based on the severity determined for the infection.

An “effective amount” or “an amount effective to treat” refers to a dose that is adequate to prevent or treat infection in an individual. Amounts effective for a therapeutic or prophylactic use will depend on, for example, the stage and severity of the disease being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations, perhaps using various rounds of administration.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the methods can provide any amount or any level of treatment or prevention of infection in a subject. Furthermore, the treatment or prevention provided by the method can include treatment or prevention of one or more conditions or symptoms of the disease being treated or prevented. Also, for purposes herein, “prevention” can encompass delaying the onset of the disease, or a symptom or condition thereof.

The following includes certain aspects of the invention.

1. A method of diagnosing infection in a subject, the method comprising:

(a) obtaining a biological sample from the subject;

(b) detecting the expression level of CD153 or CD30 in the sample; and

(c) diagnosing the subject with infection when the expression level of CD153 or CD30 in the sample is detected to be higher compared to the expression level of CD153 or CD30 in a subject without infection.

2. The method of aspect 1, wherein the expression level of CD153 is detected.

3. The method of aspect 1, wherein the expression level of CD30 is detected.

4. The method of aspect 2, wherein the higher CD153 expression level is due to expression in CD4 T cells.

5. The method of any one of aspects 1-4, wherein the biological sample is peripheral blood.

6. The method of any one of aspects 1-4, wherein the biological sample is bronchoalveolar lavage fluid.

7. The method of any one of aspects 1-6, wherein the infection is Mycobacterium tuberculosis infection (Mtb).

8. The method of aspect 7, wherein the Mtb infection is a pulmonary Mtb infection.

9. The method of any one of aspects 1-6, wherein the infection is a parasitic infection.

10. The method of aspect 9, wherein the parasite is Leishmania major or Ascaris roundworm.

11. A method of determining the latency of infection in a subject, the method comprising:

(a) obtaining a biological sample from the subject;

(b) detecting the expression level of CD153 or CD30 in the sample; and

(c) determining the infection of the subject to be latent when the expression level of CD153 or CD30 in the sample is detected to be higher compared to the expression level of CD153 or CD30 in a subject with active infection.

12. The method of aspect 11, wherein the expression level of CD153 is detected.

13. The method of aspect 11, wherein the expression level of CD30 is detected.

14. The method of aspect 12, wherein the higher CD153 expression level is due to expression in CD4 T cells.

15. The method of any one of aspects 11-14, wherein the biological sample is peripheral blood.

16. The method of any one of aspects 11-14, wherein the biological sample is bronchoalveolar lavage fluid.

17. The method of any one of aspects 11-16, wherein the infection is Mycobacterium tuberculosis infection (Mtb).

18. The method of aspect 17, wherein the sample is contacted with a Mtb antigen.

19. The method of aspect 17 or 18, wherein the Mtb infection is a pulmonary Mtb infection.

20. The method of any one of aspects 11-16, wherein the infection is a parasitic infection.

21. The method of aspect 20, wherein the parasite is Leishmania major or Ascaris roundworm.

22. A method of determining the effectiveness of a vaccine against infection in a subject, the method comprising:

(a) administering a vaccine to the subject;

(b) obtaining a biological sample from the subject;

(c) detecting the expression level of CD153 or CD30 in the sample; and

(d) determining the vaccine to be effective when the expression level of CD153 or CD30 in the sample is detected to be higher compared to the expression level of CD153 or CD30 in a subject with active infection.

23. The method of aspect 22, wherein the expression level of CD153 is detected.

24. The method of aspect 22, wherein the expression level of CD30 is detected.

25. The method of aspect 23, wherein the higher CD153 expression level is due to expression in CD4 T cells.

26. The method of any one of aspects 22-25, wherein the biological sample is peripheral blood.

27. The method of any one of aspects 22-25, wherein the biological sample is bronchoalveolar lavage fluid.

28. The method of any one of aspects 22-27, wherein the infection is Mycobacterium tuberculosis infection (Mtb).

29. The method of aspect 28, wherein the Mtb infection is a pulmonary Mtb infection.

30. The method of any one of aspects 22-27, wherein the infection is a parasitic infection.

31. The method of aspect 30, wherein the parasite is Leishmania major or Ascaris roundworm.

32. A method of determining the severity of infection in a subject, the method comprising:

(a) obtaining a biological sample from the subject;

(b) detecting the expression level of CD153 or CD30 in the sample; and

(c) determining the infection to be less severe when the expression level of CD153 or CD30 in the sample is detected to be higher compared to the expression level of CD153 or CD30 in a subject with active infection.

33. The method of aspect 32, wherein the expression level of CD153 is detected.

34. The method of aspect 32, wherein the expression level of CD30 is detected.

35. The method of aspect 33, wherein the higher CD153 expression level is due to expression in CD4 T cells.

36. The method of any one of aspects 32-35, wherein the biological sample is peripheral blood.

37. The method of any one of aspects 32-35, wherein the biological sample is bronchoalveolar lavage fluid.

38. The method of any one of aspects 32-37, wherein the infection is Mycobacterium tuberculosis infection (Mtb).

39. The method of aspect 38, wherein the Mtb infection is a pulmonary Mtb infection.

40. The method of any one of aspects 32-37, wherein the infection is a parasitic infection.

41. The method of aspect 40, wherein the parasite is Leishmania major or Ascaris roundworm.

42. A method of preventing or treating infection in a subject, the method comprising administering to the subject an effective amount of a substance that upregulates CD153 or activates CD30 in CD4 T cells.

43. The method of aspect 42, wherein the infection is Mycobacterium tuberculosis infection (Mtb).

44. The method of aspect 43, wherein the Mtb infection is a pulmonary Mtb infection.

45. The method of aspect 42, wherein the infection is a parasitic infection.

46. The method of aspect 45, wherein the parasite is Leishmania major or Ascaris roundworm.

47. The method of any one of aspects 42-46, wherein the substance upregulates CD153.

48. The method of any one of aspects 42-46, wherein the substance activates CD30.

49. A method of preventing or treating infection in a subject, the method comprising administering to the subject an effective amount of CD4 T cells induced to upregulate CD153.

50. The method of aspect 49, wherein the infection is Mycobacterium tuberculosis infection (Mtb).

51. The method of aspect 50, wherein the Mtb infection is a pulmonary Mtb infection.

52. The method of aspect 49, wherein the infection is a parasitic infection.

53. The method of aspect 52, wherein the parasite is Leishmania major or Ascaris roundworm.

54. The method of any one of aspects 49-53, wherein the CD4 T cells are taken from the subject and administered using adoptive cell transfer.

55. The method of any one of aspects 1-21 or 32-41, further comprising treating infection in a subject having infection by administering to the subject an effective amount of a substance that upregulates CD153 or activates CD30 in CD4 T cells.

56. The method of aspect 55, wherein the infection is Mycobacterium tuberculosis infection (Mtb).

57. The method of aspect 56, wherein the Mtb infection is a pulmonary Mtb infection.

58. The method of aspect 55, wherein the infection is a parasitic infection.

59. The method of aspect 58, wherein the parasite is Leishmania major or Ascaris roundworm.

60. The method of any one of aspects 55-59, wherein the substance upregulates CD153.

61. The method of any one of aspects 55-59, wherein the substance activates CD30.

It shall be noted that the preceding are merely examples of embodiments. Other exemplary embodiments are apparent from the entirety of the description herein. It will also be understood by one of ordinary skill in the art that each of these embodiments may be used in various combinations with the other embodiments provided herein.

The following example further illustrates the invention but, of course, should not be construed as in any way limiting its scope.

### Example 1

This example demonstrates certain embodiments of the invention.

### Mice

Six to twelve week old male and female B6.SJL (CD45.1) congenic, C57BL/6[TgH]EGFP:Foxp3, B6.PL-Thyla/CyJ, C57BL/6J-[KO]TCRalpha (Tcra^{-/-}), C57BL/6Tac[KO]IFNgamma N12 (Ifng^{-/-}), C57BL/6-T-bet-ZsGreen[Tg] and C57BL/6-T-bet-ZsGreen[KO]T-bet (Tbx21^{-/-}), B6.SJL-[KO] RAG1 (CD45.1+Rag1^{-/-}) mice were obtained through a supply contract between the NIAID/NIH and Taconic Farms (Rensselaer, N.Y., USA). C57BL/6-T-bet-ZsGreen[Tg] mice and C57BL/6-T-bet-ZsGreen[KO]T-bet mice were crossed to generate the Tbx21V′ mice, and a breeding colony was maintained the NIAID animal facility. Six to twelve-week-old male and female B6.129X1-Tnfsf8^{tm1Pod}/J (Tnfsf8^{-/-}) mice were purchased from The Jackson Laboratory (Bar Harbor, Me., USA), and a breeding colony was maintained at the NIAID animal facility. All animals were housed at the AAALAC International-accredited BSL3 facility at the NIAID in accordance with the National Research Council Guide for the Care and Use of Laboratory Animals. All technical procedures and experimental endpoints were approved by the National Institute of Allergy and Infectious Disease Division of Intramural Research Animal Care and Use Committee and listed in the animal study proposal LPD-24E. Mice group sizes were not determined by statistical tests and were based on the number of animals that can be housed per cage. Mice were assigned to experimental groups as available and were not randomized. The study was not performed blinded.

### Indian-Origin Rhesus Macaques

All rhesus macaques were healthy, purified protein derivative (PPD) skin test negative prior to infection. Animals were housed in an AAALAC International-accredited ABSL3 vivarium in non-human primate biocontainment racks and provided daily enrichment in accordance with the Animal Welfare Act, the Guide of the Care and Use of Laboratory Animals, and other federal statutes and regulations. Housing was also in accord with the National Institute of Allergy and Infectious Diseases Division of Intramural Research Animal Program Policy on Social Housing of Non-Human Primates (NHP). All technical procedures and experimental endpoints were approved by the National Institute of Allergy and Infectious Diseases Animal Care and Use Committee and listed in the animal study proposal LPD-25E. Euthanasia methods were in accord with the American Veterinary Medical Association Guidelines on Euthanasia. Animals ZK38, ZL43, ZK26, ZK17, ZK02 and ZJ01 were previously reported in another study (Kauffman et al., Mucosal Immunol., doi:10.1038/mi.2017.60 (2017), incorporated by reference herein in its entirety). The number of macaques used in this study (a total of 10) was not based on statistical tests and was determined based on typical group sizes in the published literature.

### Human Subjects

Study participants (n=16) were recruited from the Ubuntu Clinic, Site B in Khayelitsha (Cape Town, South Africa). Participants were divided into two groups based on their TB status: active tuberculosis (aTB, n=8) and latent tuberculosis infection (LTBI, n=8). LTBI was diagnosed based on a positive IFNγ release assay (QuantiFERON®-TB Gold In-Tube, Qiagen, Hilden, Germany), no symptoms of aTB and a negative Mtb-sputum (GeneXpert, Cepheid, Sunnyvale, Calif., USA). Diagnosis of aTB was based on clinical symptoms and/or a positive Mtb-sputum (GeneXpert). All culture positive aTB cases were fully drug sensitive and TB treatment-naïve at the time of enrollment. This work was conducted under the DMID protocol no. 15/0047. This study was approved by the University of Cape Town Human Research Ethics Committee (no. 050/2015). This study was conducted in accordance with good clinical practice (GCP) and the Declaration of Helsinki. All participants provided written informed consent.

### Mtb Infections

For mouse Mtb infections, animals were exposed to ˜100 CFU of Mtb H37Rv strain using an aerosol inhalation exposure system (Glas-Col, LLC, Terre Haute, Ind., USA). Dose calculations were measured by serial dilutions of lung homogenates on 7H11 agar plates supplemented with oleic acid-albumin-dextrose-catalase (Difco, Detroit, Mich., USA) immediately post exposure. For rhesus macaque Mtb infections, frozen bacterial stocks of known concentration were thawed and serially diluted to gain desired infection dose. Animals were then anesthetized and bacteria were instilled into the lower right lobe of the lung via bronchoscope. Dose was confirmed by plating of inoculum on agar plates.

### Intravascular Staining

Mice were injected with 2.5 μg of anti-CD45 fluorochrome-labeled antibody (30-F11), and after 3 minutes, animals were euthanized and lungs harvested for processing. For rhesus macaques, animals were anesthetized and injected with 50 μg/kg of a biotinylated anti-NHP CD45 antibody (MB4-6D6, Miltenyi Biotec, San Diego, Calif., USA). After 10 minutes, animals were exsanguinated and then euthanized. Cells were isolated from various tissues and stained with various streptavidin fluorochromes during normal staining procedures.

### Cell Sorting

Effector CD4 T cells were isolated from the lungs of Mtb infected Foxp3-EGFP mice on day 28 post-infection after administration of anti-CD45 at 2 μg per mouse by intravenous injection 3 minutes prior to euthanasia. The lungs were harvested, minced, placed into RPMI containing 1 mg/ml Collagenase D (Roche-Diagnostics, Indianapolis, Ind., USA), 1 mg/ml hyaluronidase, 50 U/ml DNase I and 1 mM aminoguanidine (all from Sigma-Aldrich, St. Louis, Mo., USA), and incubated at 37° C. for 45 minutes with shaking. The lungs were passed through a 100 μm cell strainer, and washed with PBS containing 20% fetal bovine serum (FBS). The lymphocytes were isolated by centrifugation through a density gradient of 37% Percoll from GE Healthcare Bio-Sciences (Uppsala, Sweden). The red blood cells were lysed with ACK lysis buffer (KD Medical, Columbia, Md., USA), and the cells were counted. The cells were stained with anti-CD4 (1:100), CD44 (1:100), and KLRG1 (1:100) for 30 minutes at 4° C., and then washed twice with PBS+1% FBS. The cells were stained with a viability dye Fixable Viability Dye eFluor 780 from eBioscience Inc. (San Diego, Calif., USA) for 20 minutes at 4° C., and then washed twice with PBS+1% FBS. The cells were gated on live CD44⁺Foxp3⁻CD44^{hi} cells and sorted into four populations: KLRG1⁻ivCD45^{dim}, KLRG1⁻ivCD45⁺, KLRG1⁺ivCD45^{dim}, and KLRG1⁺ivCD45⁺ on a BD Aria sorter. The cells were collected in PBS+1% FBS, and centrifuged. The cell pellets were lysed in Trizol, and stored at −80° C. until RNA isolation. This was repeated for five independent experiments, pooling the lungs of 15 to 25 mice for each sort. The RNA was isolated using the Direct-zol RNA kit (Zymoresearch, Irvine, Calif., USA) following manufacturer's instructions.

### Microarray Hybridization and Expression Analysis

Amplification and labeling of the RNA samples were performed using the Illumina TotalPrep RNA Amplification (Applied Biosystems, Foster City, Calif., USA) and an input of 500 nanograms of total RNA per sample. Biotinylated aRNA was hybridized to Illumina MouseRef-8 v2.0 Expression BeadChip (GEO Accession GPL6885) having 25,697 unique probes, using reagents provided, and imaged using the Illumina HiScan-SQ.

Signal data was extracted from the image files with the Gene Expression module (v. 1.9.0) of the GenomeStudio software (v. 2011.1) from Illumina, Inc. (San Diego, Calif., USA). Signal intensities were converted to log 2 scale. Calculation of detection p-values is described in the GenomeStudio Gene Expression Module User Guide. Data for array probes with insufficient signal (detection p-value <0.1 in at least 2 arrays) were considered “not detected” and were removed from the dataset.

After dropping undetected probes, quantile normalization was applied across all arrays. ANOVA was performed on the normalized log 2 expression estimates to test for mRNA expression differences for 10 comparisons: four comparisons for Effectors vs Naïve in the same compartment (Effector KLGR1⁺ or KLGR1⁻ vs. Naïve in either IV⁺ or IV⁻ compartments) and six pairwise comparisons between four Effector cell types (all permutations of iv⁺ or iv⁻ and KLGR1⁺ or KLGR1⁻). A p-value of 0.05 was used for the statistical significance cutoff, after adjusting for the familywise error rate (FWER) using Benjamini-Hochberg method to account for multiple testing. Statistical analysis was performed using JMP/Genomics software version 6.0 (SAS Institute Inc., Cary, N.C., USA).

Hierarchical clustering (Ward method) utilized standardized average signal (log 2) by cell type. For genes with multiple probes, representative probes were chosen as the one with the maximum average signal per gene across all cell types. Genes were considered as members of the TNF superfamily (TNFSF) or TNF receptor superfamily (TNFRSF) if the gene name appeared in the HUGO Gene Family for “Tumor necrosis factor superfamily” or “Tumor necrosis factor receptor superfamily” with additional mouse representatives for genes that appeared in both the SMART category for TNFR (SM00208) and the GO category of “death receptor activity” for Mus musculus. Among genes represented on the MouseRef-8 v2.0 array, 28 were annotated as members of TNFRSF and 17 as members of TNFSF.

### CD4 T Cell Adoptive Transfers

Mouse adoptive transfers were performed by isolating CD4 T cells from naïve WT, Ifng^{-/-}, and Tnfsf8^{-/-} mice. Spleens and lymph nodes were harvested from each and mashed through a 100 um cell strainer. After ACK red blood cell lysis, CD4 T cells were positively selected using MACS magnetic beads and columns (Miltenyi Biotec, San Diego, Calif., USA). RAG1^{-/-} or Tcra^{-/-} recipients were reconstituted with between 3.5×10⁶ and 4.2×10⁶ purified CD4 T cells of each indicated population depending on the experiment. Purified CD4 T cells were injected into the recipients either 1 day prior to or 7 days post Mtb infection, depending on the experiment.

### Cell Isolations, Peptide Stimulations, and Flow Cytometry

Mice lungs were harvested and minced using a gentleMACs dissociator (Miltenyi Biotec, San Diego, Calif., USA) and were enzymatically digested in a shaker incubator at 37° C. for 45 minutes in RPMI containing 1 mg/ml Collagenase D (Roche-Diagnostics, Indianapolis, Ind., USA), 1 mg/ml hyaluronidase, 50 U/ml DNase 1, and 1 mM aminoguanidine (all from Sigma Aldrich, St. Louis, Mo., USA). Suspensions were then passed through a 100 um cell strainer and enriched for lymphocytes using a 37% Percoll density gradient centrifugation. Cells were stimulated in complete medium containing 10% FBS at 1×10⁷/ml at 37° C. for 5 hours with either ESAT-6₁₋₂₀ or TB10.4₄₋₁₁ in the presence of Brefeldin-A, monensin, and 1 mM aminoguanidine. Tetramer stains were performed by incubating 1×10⁶ cells with a 1:50 dilution of I-A^{b} ESAT-6₄₋₁₇ in complete medium containing 10% FBS, 1 mM aminoguanidine, and monensin. Tetramers were produced by the NIAID tetramer core facility (Emory University, Atlanta, Ga., USA). After stimulation or tetramer stains, cells were stained with various combinations of the following fluorochrome-labeled antibodies: CD4 (RM4-4), CD8 (53-6.7), CD44 (IM7), KLRG1 (2F1/KLRG1), TNF (MP6-XT22), IFNγ (XMG1.2), CD153 (RM153), Foxp3 (FJK-16s), GITR (YGITR 765), OX-40 (OX-86), RANKL (IK22/5), CD154 (MR1), CD30 (mCD30.1), and Fixable Viability Dye eFluor 780, all purchased from Biolegend (San Diego, Calif., USA), eBioscience (San Diego, Calif., USA), BD Biosciences (San Jose, Calif., USA), or R&D Systems (Minneapolis, Minn., USA).

To isolate cells from rhesus macaque tissues at necropsy, lungs and lymph nodes were resected and homogenized using a gentleMACS dissociator (Miltenyi Biotec, San Diego, Calif., USA). Consolidation-like lesions at the site of bacterial instillation were homogenized and enzymatically digested using a gentleMACS dissociator in RPMI-1640 medium supplemented with 1 mg/ml Collagenase D (Roche-Diagnostics, Indianapolis, Ind., USA), 1 mg/ml hyaluronidase and 50 U/ml DNase 1 (both from Sigma Aldrich, St. Louis, Mo., USA). All homogenates were then passed through a 100 μm cell strainer and enriched for lymphocytes using a 25%/50% Percoll density gradient centrifugation. Granulomas were simply mashed through a 100 μm cell strainer. Blood and BAL were also collected from the animals at various time points during the studies. Peripheral blood mononuclear cells were isolated from whole blood by 90% Ficoll-paque PLUS gradient separation (GE Healthcare Biosciences, Pittsburgh, Pa., USA). Bronchoalveolar lavage (BAL) samples were taken by inserting tubing into the trachea with assistance by a largynoscope, instilling sterile saline into the lungs and immediately aspirating. BAL samples were passed through a 100 μm cell strainer to remove any debris and then cells isolated for assays by centrifugation. For T cell stimulations, cells were incubated in X-Vivo 15 media supplemented with 10% FBS for 6 hours at 37° C. with either MTB300 peptide pool (2 μg/ml) or ESAT-6/CFP-10 peptide pools (1 μg/ml), all in the presence of brefeldin-A and monensin. They were then stained with various combinations of the following fluorochrome-labeled antibodies: CD3 (SP34-2), CD4 (OKT4), CD8 (RPA-T8), TNF (Mab11), IFNγ (4S.B3), CD153 (116614), CD30 (BerH8) and Fixable Viability Dye eFluor 780, all purchased from Biolegend, eBioscience (San Diego, Calif., USA), BD Biosciences (San Jose, Calif., USA), and R&D Systems (Minneapolis, Minn., USA). Data for all mouse and macaque samples were collected on a BD LSRfortessa and analyzed using FlowJo software (version 10.0.8, Tree Star, Ashland, Oreg., USA).

For human PBMC analysis, heparinized whole blood was incubated at 37° C. for 5 hours with a MTB300 peptide megapool (1.5 μg/ml; see below) in the presence of anti-CD28 and anti-CD49d antibodies (1 ug/ml) and Brefeldin-A (10 μg/ml). After incubation, red blood cells were lysed, cells were then stained with a fixable near-infra red viability dye, fixed using eBioscience Foxp3 fixation buffer for 30 min at room temperature, and cryopreserved in freezing media containing 50% FCS, 40% RPMI and 10% DMSO. Cells were stored at −80° C. until usage. Cryopreserved cells were thawed, washed and incubated 10 min in the eBioscience Foxp3 Perm/Wash buffer. Cells were then stained for 45 min at 4° C. using the following antibodies: CD3 BV650 (OKT3, Biolegend), CD4 PerCPcy5.5 (OKT4, Biolegend), CD8 BV510 (RPA-T8, Biolegend), HLA-DR BV605 (LN3, eBioscience), CD153 PE (116614, R&D), KLRG1 PE-vio770 (REA261, Miltenyi), IFNγBV711 (4S.B3, Biolegend), TNF FITC (Mab11, Biolegend) and IL-2 BV421 (MQl-17H12, Biolegend). Cells were acquired on a BD LSR-II and data analyzed using FlowJo and Pestle and SPICE. A positive cytokine response was defined as three-fold above background.

### Statistical Analysis

Prism (version 7, Graphpad Software, La Jolla, Calif., USA) and SPICE were used to perform all statistical analyses. The statistical difference between experimental groups was determined by unpaired Student's t-tests or Mann-Whitney U-tests, one-way analysis of variance with Fisher's least significant difference test for multiple comparisons, and log-rank test for survival studies. A P value of <0.05 was considered significant.

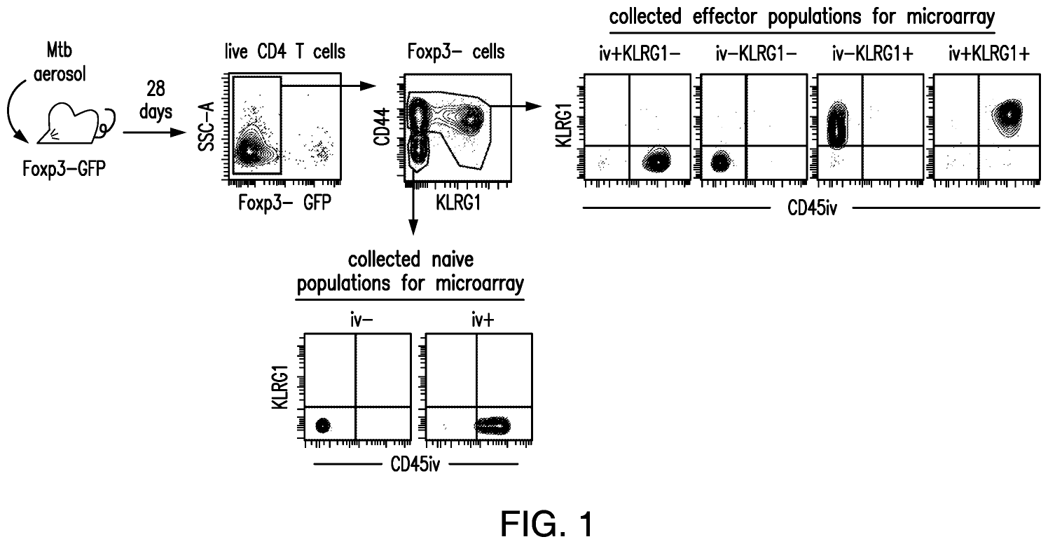

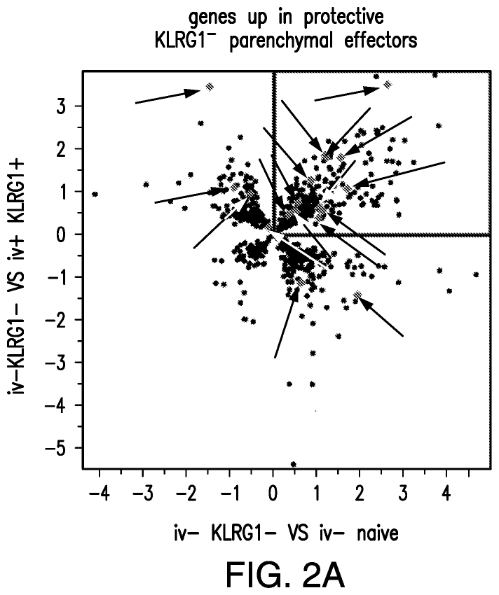

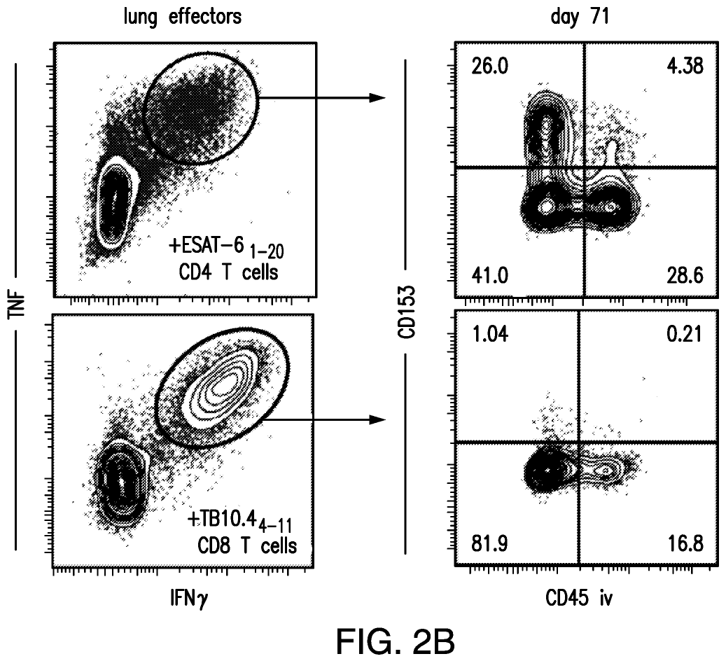

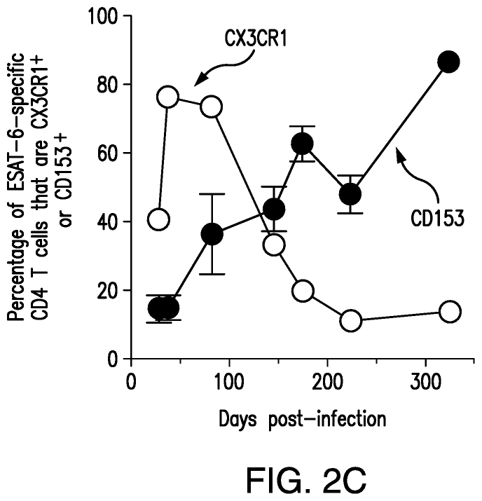

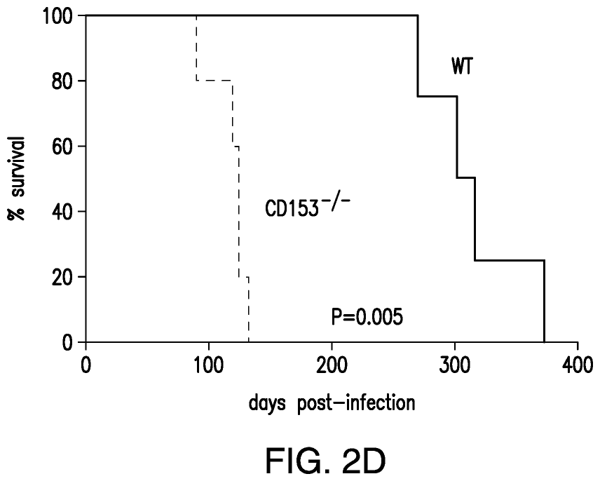

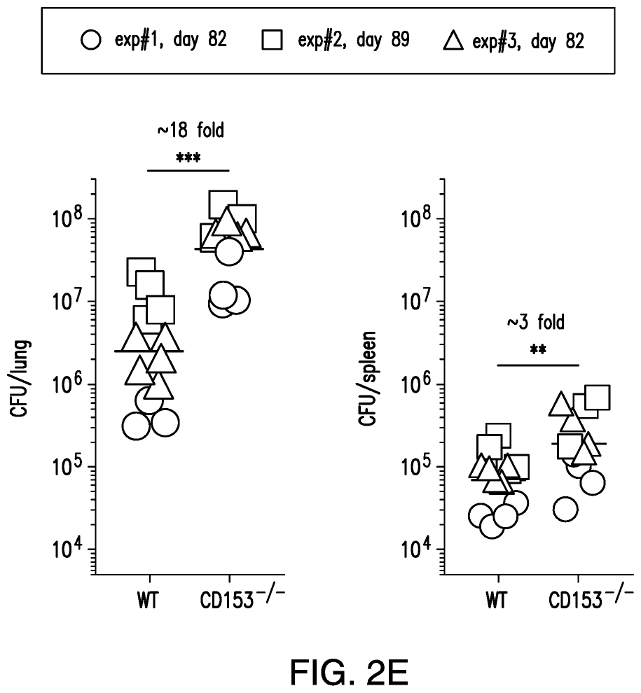

It has been previously shown that KLRG1⁻CX3CR1⁻ effector CD4 T cells are able to migrate into the lung parenchyma and adoptively transfer protection against Mtb infection, whereas terminally-differentiated KLRG1⁺CX3CR1⁺CD4 T cells accumulate in the lung blood vasculature and do not protect. To identify molecules selectively associated with host-protective CD4 T cells, a comparison was made of the gene expression pattern of CD44^{high}Foxp3⁻GFP⁻ lung effector cells from Mtb-infected mice that were separated through fluorescence-activated cell sorting (FACS) into four populations based on KLRG1 expression and intravascular localization (FIG. 1). CD44^{low} Foxp3⁻GFP⁻ naïve T cells purified from the lung parenchyma and vasculature served as respective controls. It was hypothesized that genes of interest would be significantly upregulated in the most abundant and highly protective effector subset (that is lung parenchymal CD45 intravascular stain negative (CD45iv⁻) and KLRG1⁻ cells) compared to both naïve T cells and the most abundant non-protective subset (that is, CD45 intravascular stain positive (CD45iv⁺) and KLRG1⁺ cells). There were identified 211 genes with statistically significant expression differences by both these pairwise comparisons (FIG. 2A). Gene ontology (GO) enrichment analysis found that TNF and TNF receptor (TNFR) superfamily members accounted for >5% of all microarray probes for genes with high expression in protective effector CD4 T cells, corresponding to a ˜16-fold enrichment compared to the frequency of this class among all genes measured on the microarray (Fisher's Exact test p<0.0001). Because TNF(R) superfamily molecules are potent mediators of inflammatory responses, the expression patterns of TNF(R) superfamily molecules were examined across all six populations of T cells and identified genes for which the expression was significantly different for any comparison. KLRG1⁻ parenchymal effectors and KLRG1⁺ intravascular effectors showed different patterns of expression of these TNF(R) superfamily molecules (FIG. 2A), with the majority being expressed to a much greater extent in parenchymal effector CD4 T cells.

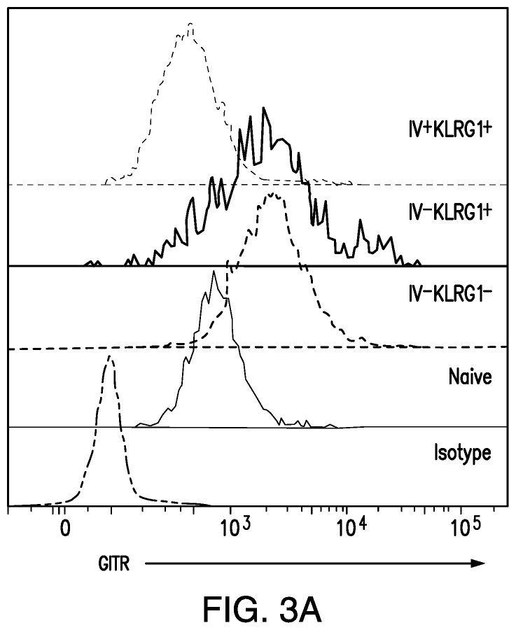

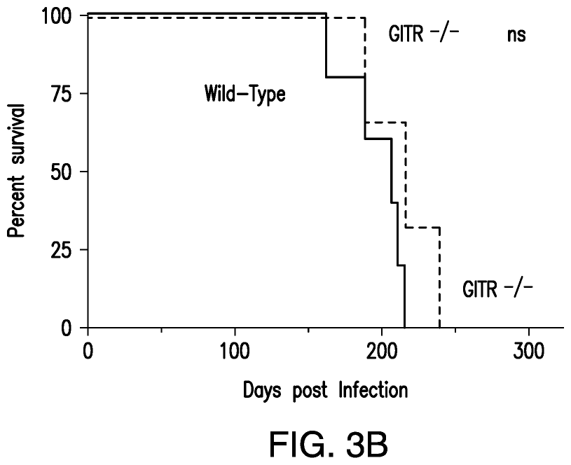

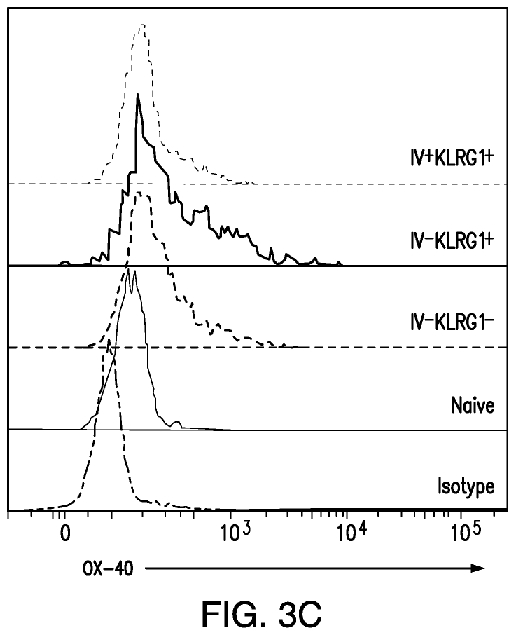

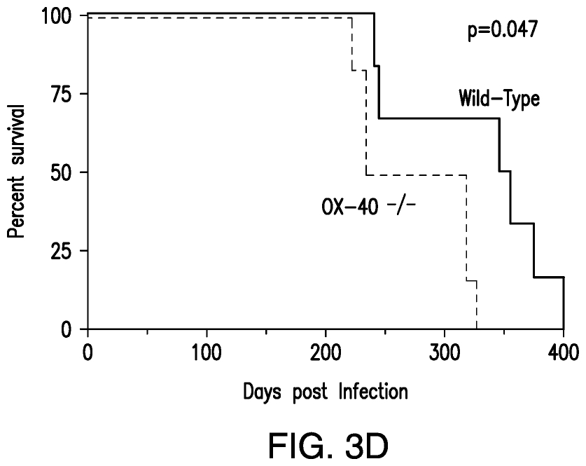

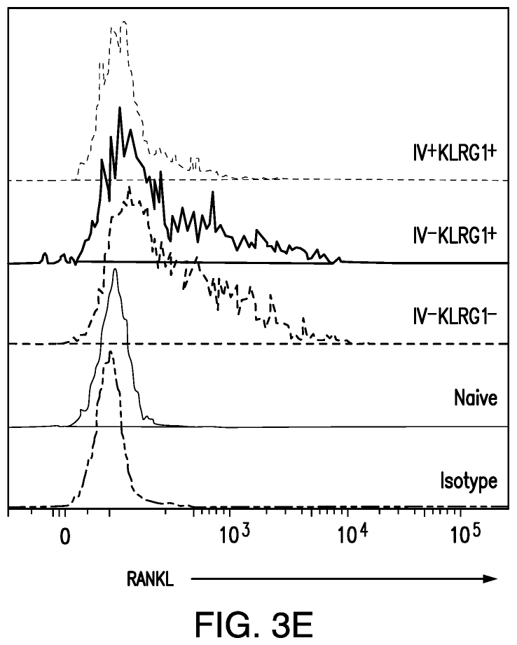

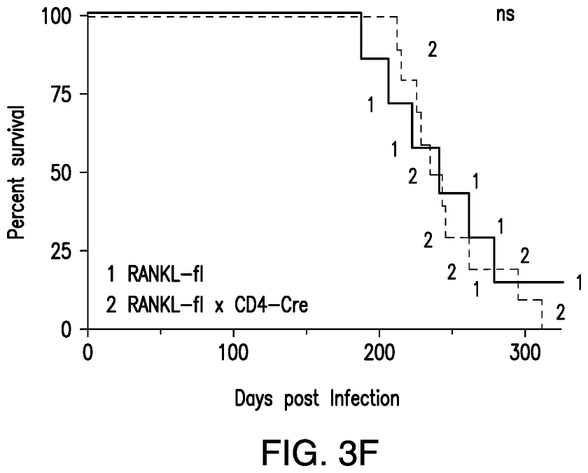

Tnfsf5 (which encodes CD40L), Tnfsf14 (which encodes LIGHT), Ltα (which encodes LTα) and Tnfrsf9 and Tnfsf9 (which encode 4-1BB and 4-1BB ligand, respectively) were all preferentially expressed by protective CD4 T cells, but each of these pathways has been previously shown to have little to no role in control of Mtb infection in mice. The microarray analysis also found that Tnfrsf18 (which encodes GITR), Tnfrsf4 (which encodes OX40) and Tnfsf11 (which encodes RANKL) were preferentially expressed by the protective effector CD4 T cells, and their presence was confirmed by flow cytometry (FIGS. 3A-3F). The role of each of these pathways was tested in host survival following Mtb infection, and it was found that Tnfsf18^{-/-}, Tnfrsf4^{-/-} and Tnfsf11^{f1/f1}Cd4^{cre} mice all displayed survival times similar to their wild-type (WT) controls. Therefore, each of these molecules were also not essential for control of Mtb infection (FIGS. 3A-3F).

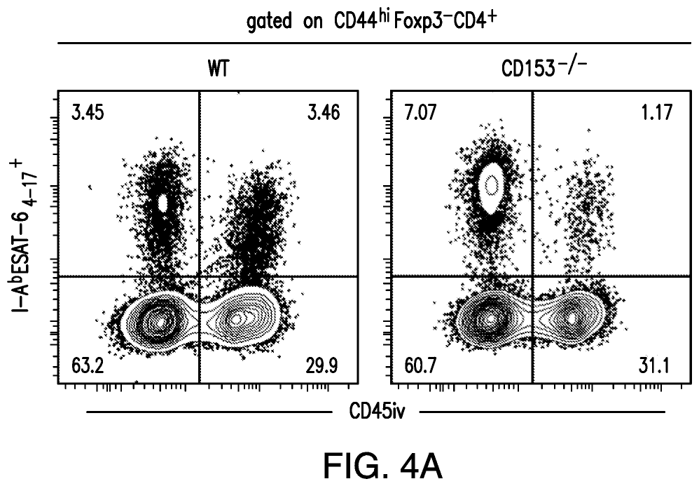

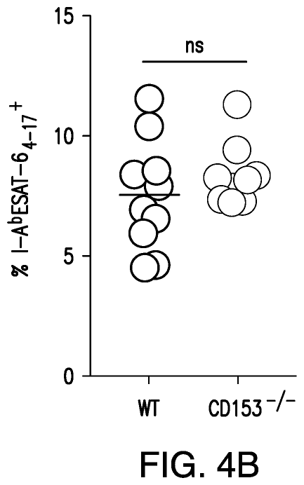

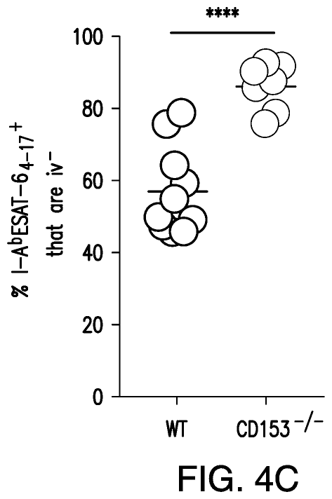

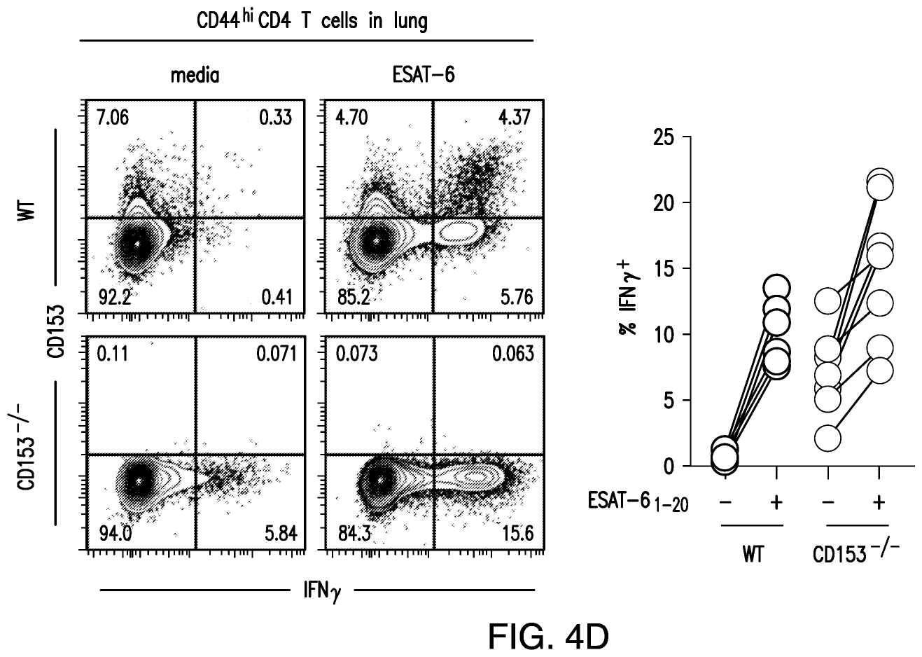

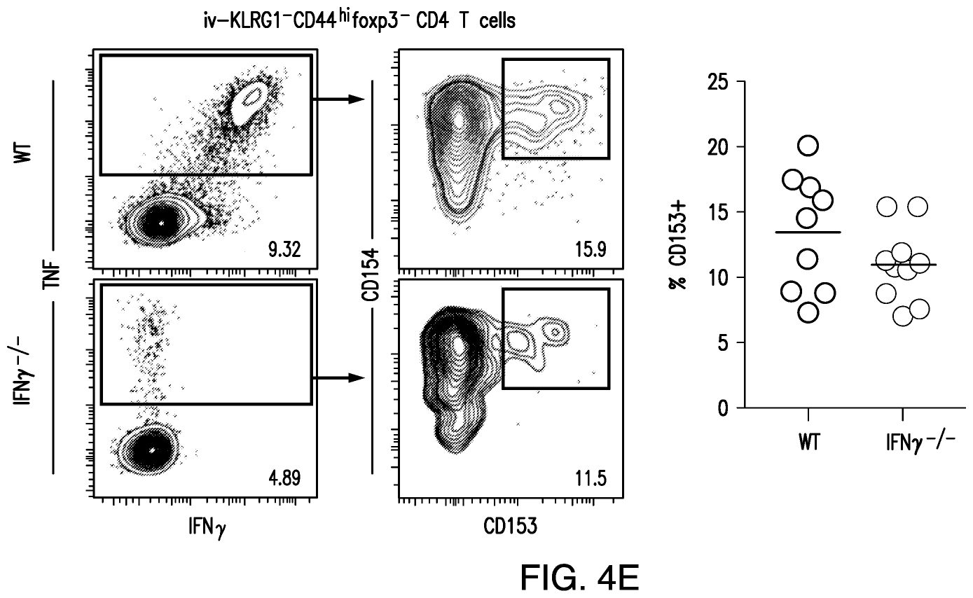

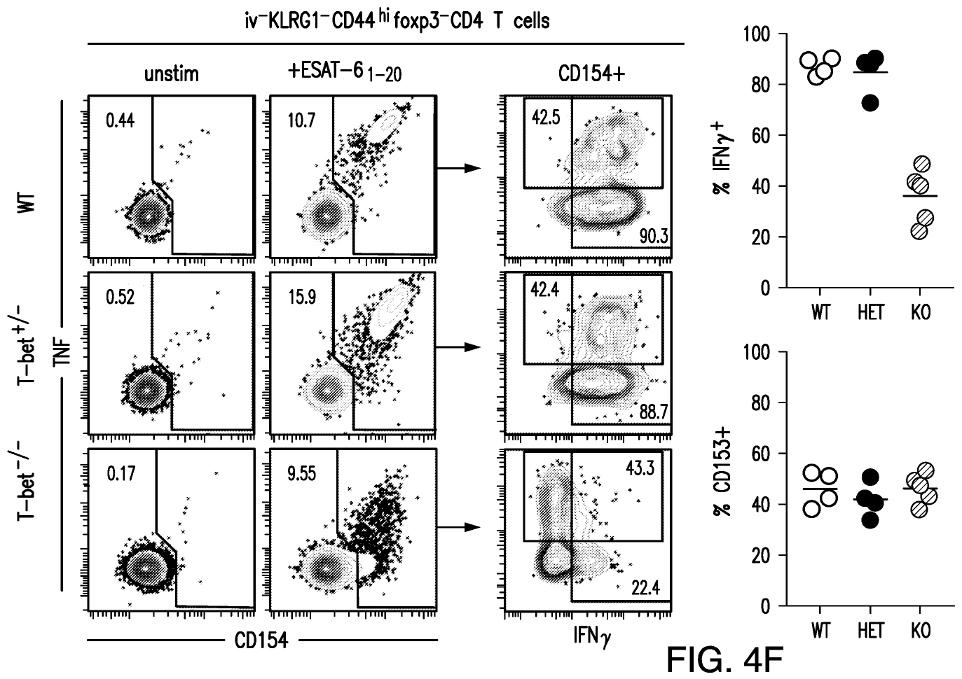

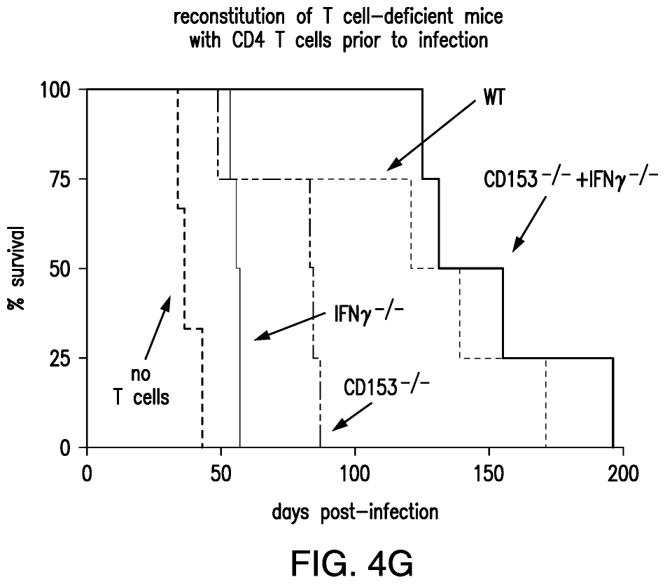

Tnfsf8 (which encodes CD153) gene expression was also significantly higher in host-protective effector cells compared to both naïve and non-protective CD4 T cells. Consistent with the microarray data, in Mtb-infected mice CD153 was detected by flow cytometry on restimulated parenchymal CD4 T cells specific for the mycobacterial peptide antigen ESAT-6₁₋₂₀ (FIG. 2B). The expression of CD153 was similar between Mtb-specific CD4 T cells in the lung tissue parenchyma and bronchoalveolar lavage (BAL) fluid, and CD153 was not detected on Foxp3⁺ regulatory T cells in the lung. CD8 T cells specific for the Mtb-derived peptide TB10.4₄₋₁₁ did not express CD153 (FIG. 2B), indicating that Mtb-specific CD4 T cells, but not CD8 T cells, upregulate CD153 after restimulation. Interestingly, CD153 expression by lung Mtb-specific CD4 T cells steadily increased during the >300-day course of infection studied (FIG. 2B), perhaps reflecting the gradual loss of terminal effector cells that do not express CD153 (FIG. 2C). Strikingly, Tnfsf8^{-/-} mice succumbed early following low-dose aerosol exposure compared to WT controls, indicating that this molecule plays an important role in host resistance to Mtb infection (FIG. 2D). At day 28 post-infection there was no difference in the bacterial loads in the lungs of WT and Tnfsf8^{-/-} mice (WT, log₁₀ 5.64±0.1 c.f.u. versus knockout (KO), log₁₀ 5.60±0.17 c.f.u.). However, approximately 80-90 days post-infection, bacterial loads were ˜10-30-fold higher in the lungs but only ˜3-fold higher in the spleen of Tnfsf8^{-/-} compared to WT mice (FIG. 2E). Staining for acid fast bacilli found that infected cells in the lungs of Tnfsf8^{-/-} mice contained greatly increased numbers of bacilli per infected cell compared to WT. Therefore, unlike Ifng^{-/-} mice that display greater fold increases in splenic compared to the lung bacterial loads, Tnfsf8^{-/-} mice primarily show a defect in control of pulmonary Mtb infection. The expansion of I-A^{b}/ESAT-6₄₋₁₇-specific CD4 T cells was similar in the lungs of Tnfsf8^{-/-} and WT mice (FIGS. 4A and 4B). There was a slight increase in the percentage of I-A^{b}/ESAT-6₄₋₁₇-specific CD4 T cells that were localized to the lung parenchyma in Tnfsf8^{-/-} mice relative to WT controls, perhaps reflecting the increased bacterial loads (FIGS. 4A and 4C). Therefore, CD153 is not required for the expansion or migration of Mtb-specific CD4 T cells into the lungs. Moreover, Mtb-infected Tnfsf8^{-/-} mice displayed normal frequencies of peptide-specific IFNγ-producing CD4 T cells in the lungs (FIG. 4D), indicating that CD153 is also not required for Th1 differentiation during Mtb infection. In the lungs of Ifng^{-/-} mice, no difference was found in CD153 expression by Mtb-specific TNF⁺CD154⁺ parenchymal KLRG1 effector cells compared to WT controls (FIG. 4E), indicating that IFNγ production is not required for induction of CD153. CD153 expression on CD4 T cells in Mtb-infected Tbx21^{+/+} (which encodes T-bet), Tbx21^{+/-} and Tbx21^{-/-} mice. was compared. Tbx21^{+/-} and Tbx21^{-/-} mice do not generate KLRG1⁺ intravascular effector cells, so following peptide restimulation gating was done on TNF⁺CD154⁺KLRG1⁻CD44^{high}Foxp3⁻ effector CD4 T cells to directly compare Ag-specific T cells in similar differentiation states in each mouse strain. IFNγ expression was defective in Tbx21^{+/+} mice (FIG. 4E). However, CD153 expression by Mtb-specific KLRG1⁻ effector CD4 T cells was identical in all three mouse strains (FIG. 4F). Collectively, these data show that CD153 is not required for Th1 polarization, and Th1 polarization is not required for CD153 expression.

To determine whether CD153 production specifically by CD4 T cells plays a role in host resistance to Mtb infection, T cell-deficient mice were reconstituted with different combinations of WT and knockout (KO) T cells. T-cell-deficient mice that received either Ifng^{-/-} or Tnfsf8^{-/-} CD4 T cells succumbed earlier to infection compared to recipients of WT T cells, and mice that received Ifng^{-/-} T cells were the most susceptible (FIG. 4G). Although Ifng^{-/-} and Tnfsf8^{-/-}CD4 T cells failed to transfer normal levels of protection when injected separately, reconstitution of T cell deficient hosts with a 1:1 mixture of Ifng^{-/-} and Tnfsf8^{-/-}CD4 T cells protected mice as well as transfer of WT CD4 T cells (FIG. 4G). Tnfsf8^{-/-}CD4 T cells are otherwise able to mediate protection to Mtb infection, given another source of CD153 signals is present, such as the Ifng^{-/-}CD4 cells here. These data indicate that, in addition to IFNγ, CD4 T cells require CD153 to mediate protection against Mtb infection. This particular experimental design also revealed that CD153 and IFNγ need not be expressed on the same CD4 T cells in order to protect against Mtb infection. However, the data show that Mtb-specific CD153-expressing CD4 T cells also express IFNγ, so it is most likely that during Mtb infection of intact mice, both CD153 and IFNγ can be co-delivered by the same CD4 T cell. It may also be that CD153 expression by other cell types may also be important for control of Mtb infection.

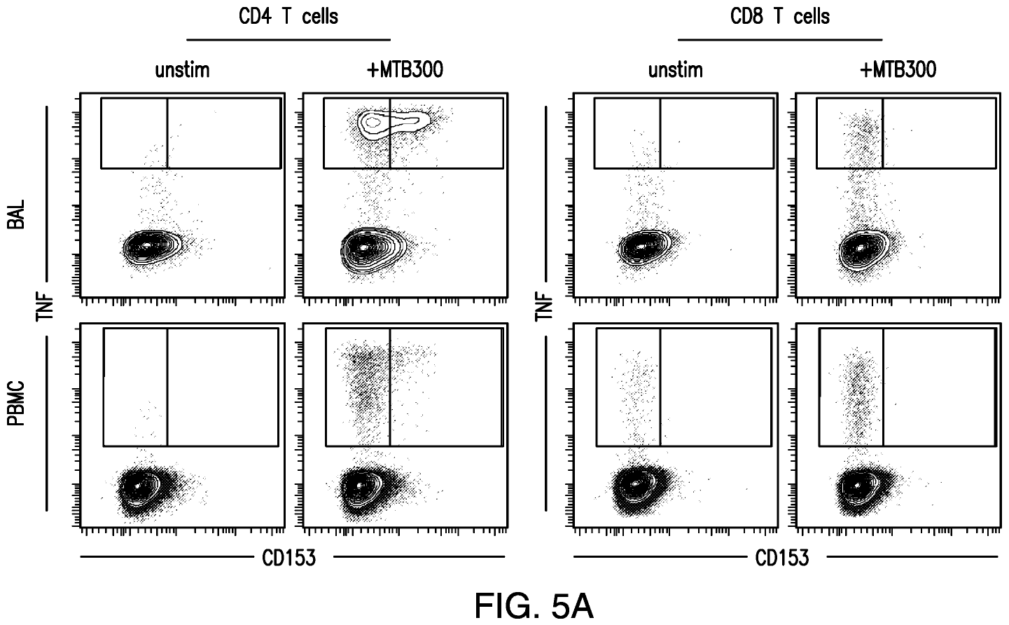

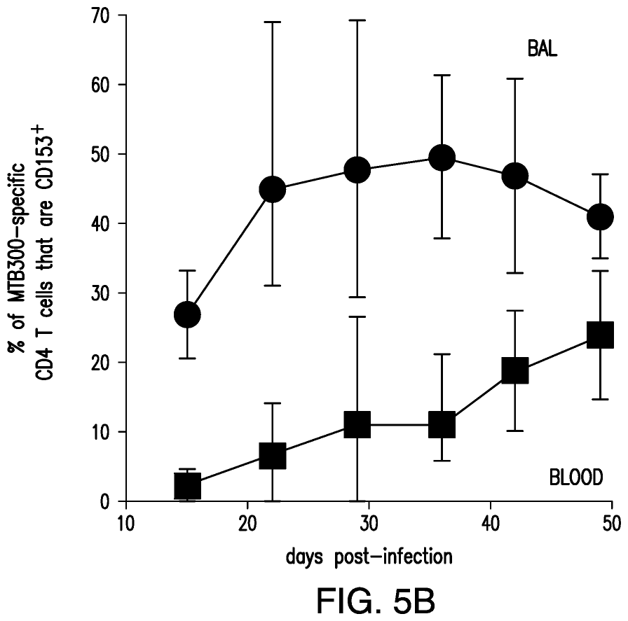

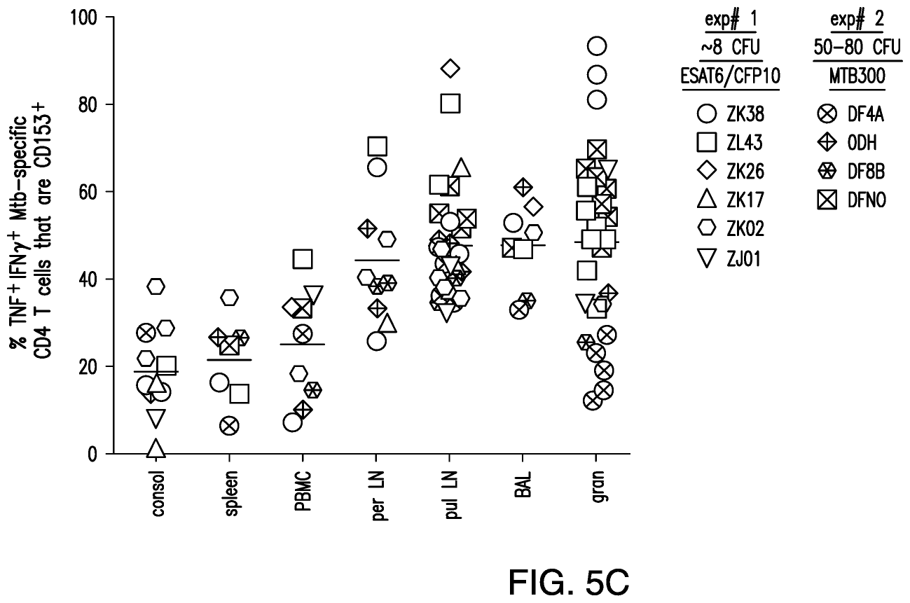

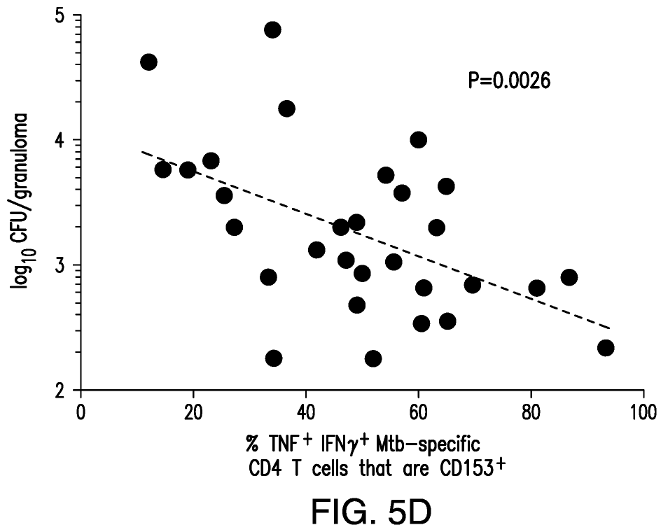

CD153 expression by T cells during Mtb infection of rhesus macaques was then examined. Mtb-specific CD4 T cells were analyzed in the airways and blood after restimulation with either a pool of two immunodominant antigens (ESAT-6 and CFP-10) or a pool of 300 Mtb-derived peptides (MTB300 megapool) (Mothe et al., Tuberculosis (Edinb,), 95: 722-735, (2015) and Lindestam Arlehamn et al., PLoS Pathog., 12, e1005760 (2016), each incorporated by reference herein in its entirety). Following restimulation, Mtb-specific CD4 T cells, but not CD8 T cells, from the BAL and blood expressed CD153, and significantly more CD4 T cells in the BAL expressed CD153 compared to the blood at each time point analyzed (FIGS. 5A and 5B). At necropsy, it was found that CD153 was the lowest on restimulated Mtb-specific CD4 T cells in the blood, spleen, and in consolidation-like inoculation site lesions in the lungs—that is unorganized sites of high bacterial burden (FIG. 5C and Table 1).

TABLE 1

| Tukey's | | | | | |
|---|---|---|---|---|---|
| multiple | | | | | |
| comparisons | Mean | 95.00% CI | Signif- | | Adjusted |
| test | Diff | of diff. | icant? | Summary | P Value |
| per LN vs. | −3.322 | −19.88 to | No | ns | 0.9966 |
| pul LN | | 13.23 | | | |
| per LN vs. | 19.27 | −1.645 to | No | ns | 0.0916 |
| PBMC | | 40.19 | | | |
| per LN vs. | −3.495 | −25.09 to | No | ns | 0.9990 |
| BAL | | 18.1 | | | |
| per LN vs. | −4.131 | −20.69 to | No | ns | 0.9888 |
| gran | | 12.42 | | | |
| per LN vs. | 25.58 | 5.687 to | Yes | ** | 0.0036 |
| consol | | 45.47 | | | |
| per LN vs. | 22.81 | 0.377 to | Yes | * | 0.0436 |
| spleen | | 45.24 | | | |
| pul LN vs. | 22.59 | 5.355 to | Yes | ** | 0.0028 |
| PBMC | | 39.83 | | | |
| pul LN vs. | −0.1734 | −18.23 to | No | ns | >0.9999 |
| BAL | | 17.88 | | | |
| pul LN vs. | −0.8097 | −12.37 to | No | ns | >0.9999 |
| gran | | 10.75 | | | |
| pul LN vs. | 28.9 | 12.92 to | Yes | **** | <0.0001 |
| consol | | 44.87 | | | |
| pul LN vs. | 26.13 | 7.083 to | Yes | ** | 0.0014 |
| spleen | | 45.18 | | | |
| PBMC vs. | −22.77 | −44.88 to | Yes | * | 0.0394 |
| BAL | | −0.6458 | | | |
| PBMC vs. | −23.4 | −40.64 to | Yes | ** | 0.0017 |
| gran | | −6.165 | | | |
| PBMC vs. | 6.306 | −14.15 to | No | ns | 0.9672 |
| consol | | 26.77 | | | |
| PBMC vs. | 3.54 | −19.4 to | No | ns | 0.9992 |
| spleen | | 26.48 | | | |
| BAL vs. | −0.6363 | −18.69 to | No | ns | >0.9999 |
| gran | | 17.42 | | | |
| BAL vs. | 29.07 | 7.92 to | Yes | ** | 0.0014 |
| consol | | 50.22 | | | |
| BAL vs. | 26.31 | 2.746 to | Yes | * | 0.0184 |
| spleen | | 49.86 | | | |
| gran vs. | 29.71 | 13.73 to | Yes | **** | <0.0001 |
| consol | | 45.68 | | | |
| gran vs. | 26.94 | 7.892 to | Yes | *** | 0.0009 |
| spleen | | 45.99 | | | |
| consol vs. | −2.766 | −24.78 to | No | ns | 0.9998 |
| spleen | | 19.24 | | | |

By contrast, CD153 expression by Mtb-specific CD4 T cells was relatively higher in the BAL and lymph nodes. In individually resected granulomas, there was a broad distribution in the percentage of Mtb-specific CD4 T cells that expressed CD153 (FIG. 5C). The level of CD153 expression on Mtb-specific T cells inversely correlated with bacterial loads in these isolated lesions (FIG. 5D). Thus, similar to mice, Mtb-specific CD4 T cells expressing CD153 are preferentially found in the lungs compared to blood. Moreover, the presence of CD153 expressing CD4 T cells correlates with better bacterial control in individual granulomas.

It was next asked whether CD153 expression by Mtb-specific CD4 T cells correlates with latent or active disease in Mtb-infected humans. Peripheral blood T cells were analyzed from a cohort of eight healthy individuals with controlled latent Mtb infection and 8 individuals with active tuberculosis (TB) in Cape Town, South Africa (patient characteristics in Table 2).

TABLE 2

| | | | | | baseline | baseline | | |
|---|---|---|---|---|---|---|---|---|
| | | | | baseline | sputum | sputum | | |
| | | | | sputum | liquid | culture | | |
| | TB | | | smear | culture | TTP | Previous | IGRA |
| PID | status | Age | gender | grade | result | (days) | TB | (IU/ml) |
| 1008 | Active | 23 | M | 2+ | pos | 11 | No | nd |
| 1009 | Active | 18 | M | 3+ | pos | 6 | No | nd |
| 1012 | Active | 34 | M | neg | neg | na | Yes | nd |
| 1017 | Active | 48 | M | 3+ | pos | 5 | Yes | nd |
| 1019 | Active | 21 | M | 1+ | pos | 6 | No | nd |
| 1020 | Active | 29 | M | 1+ | pos | 8 | unkown | nd |
| | | | | (scanty) | | | | |
| 1021 | Active | 24 | M | 3+ | nd | nd | No | nd |
| 1024 | Active | 22 | M | 3+ | pos | 4 | unkown | nd |
| 2005 | Latent | 31 | M | na | na | na | No | 3.24 |
| 2071 | Latent | 22 | F | na | na | na | No | 1.19 |
| 2079 | Latent | 31 | F | na | na | na | No | >10 |
| 2091 | Latent | 21 | M | na | na | na | No | 1.51 |
| 2111 | Latent | 27 | F | na | na | na | No | 4.21 |
| 2159 | Latent | 30 | F | na | na | na | No | 3.26 |
| 2167 | Latent | 25 | F | na | na | na | No | 2.39 |
| 2226 | Latent | 50 | M | na | na | na | No | 7.35 |

na: not applicable, nd: not done
Note:
Previous Tb episodes (102 and 107 ) Occurred more than 10 years prior to enrolement (1997 and 2002, repectively)

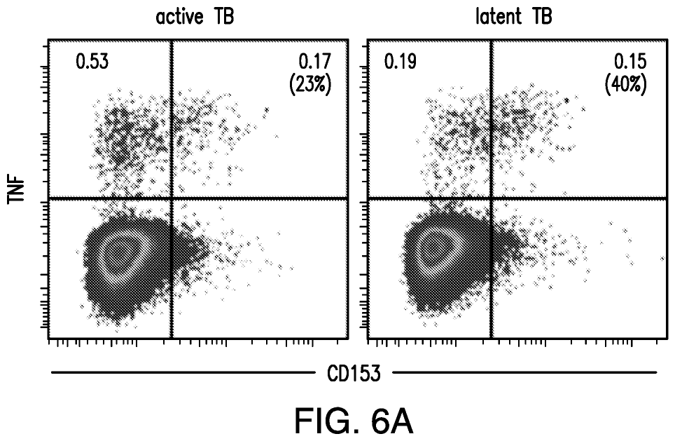

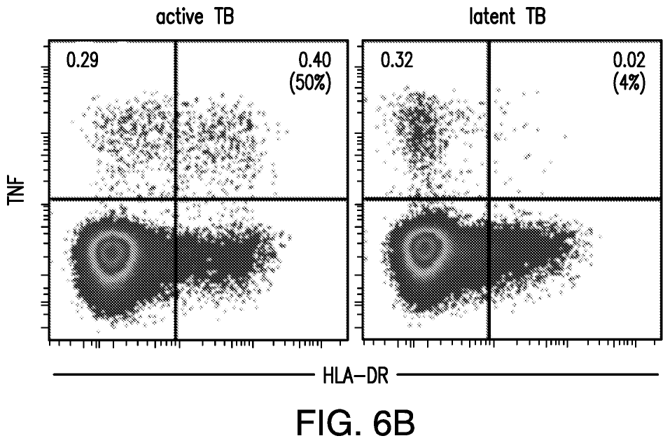

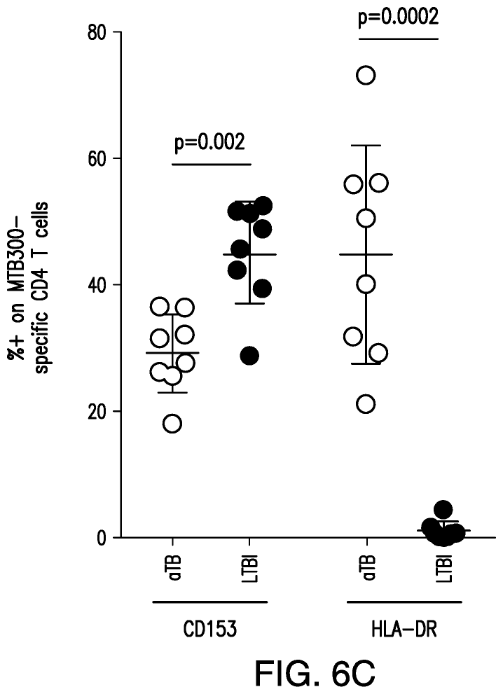

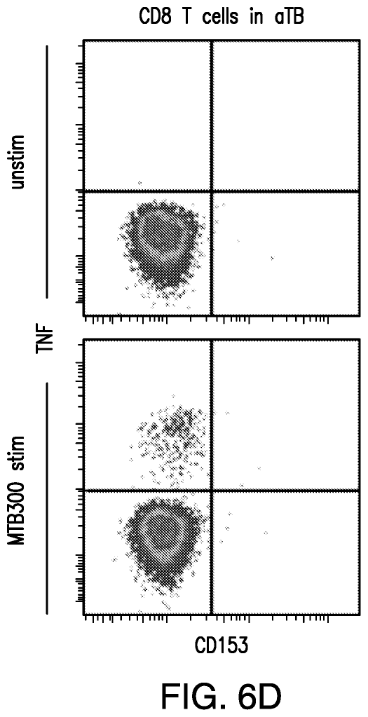

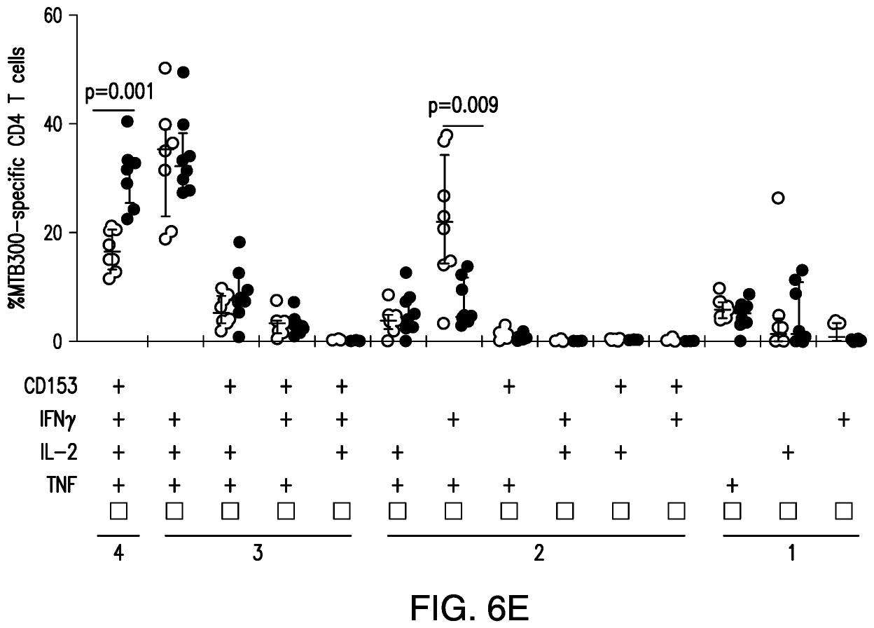

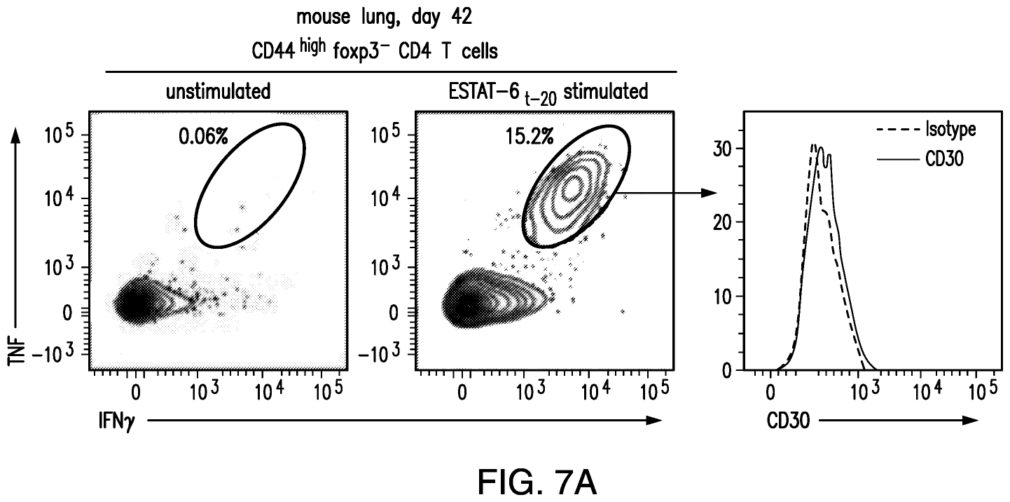

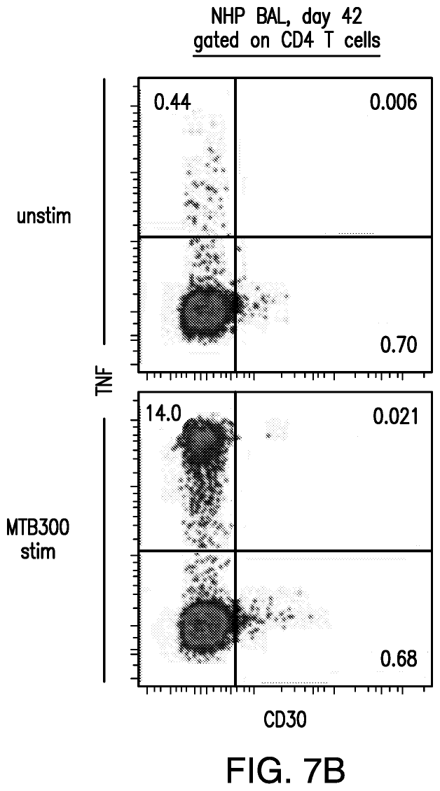

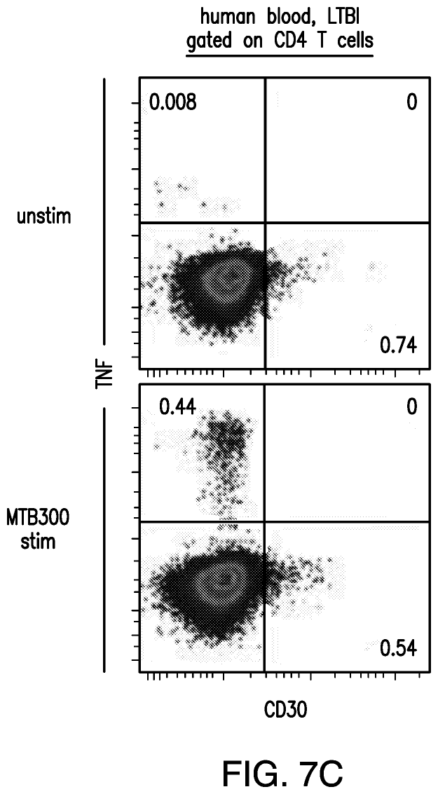

Following restimulation with the MTB300 megapool, CD153 was expressed on human Mtb-specific CD4 T cells and was significantly higher in individuals with latent Mtb infection compared to patients with active TB (FIGS. 6A and 6C). It has previously been shown that Mtb-specific CD4 T cells upregulate the activation marker HLA-DR during active disease, so HLA-DR expression was examined together with CD153. Increased expression of HLA-DR on Mtb-specific CD4 T cells was observed in individuals with active compared to latent Mtb infection (FIGS. 6B and 6C). As seen in mice and macaques, human Mtb-specific CD8 T cells did not express CD153, further indicating that this is a helper and not killer T cell pathway (FIG. 6D). Moreover, CD30, the receptor for CD153, was not detected on Mtb-specific CD4 T cells in mice, rhesus macaques or humans (FIGS. 7A-7C). Overall, increased CD153 expression by Mtb-specific CD4 T cells correlates with controlled latent Mtb infection in humans.

Elevated frequencies of polyfunctional CD4 T cells have been shown to be higher during latent compared to active TB in humans, so next examined were the co-expression of CD153 with IFNγ, TNF, and IL-2. CD153 expression was largely restricted to a subset of highly polyfunctional CD4 T cells co-producing IFNγ, TNF and IL-2, and this quadruple producing subset was significantly elevated in individuals with latent Mtb infection compared to those with active TB (FIG. 6E). Interestingly, the frequency of triple producing cells making IFNγ, TNF, and IL-2 that were negative for CD153 was similar between the two patient groups. T cells that were less polyfunctional, producing only IFNγ and TNF were elevated in active TB compared to latent infection (FIG. 6E). Thus, it is possible that the previously described loss of polyfunctional cells during active TB in humans reflects, at least in part, the specific loss of CD153 producing CD4 T cells.

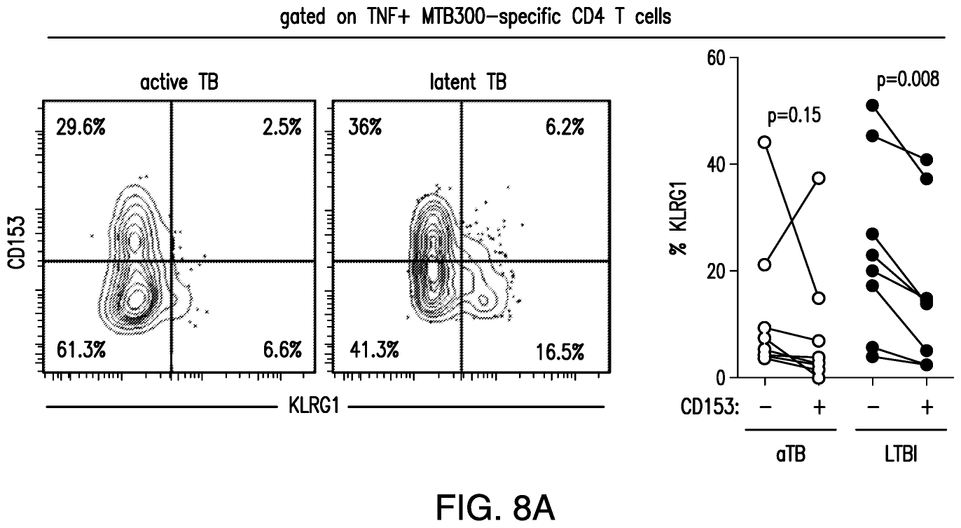

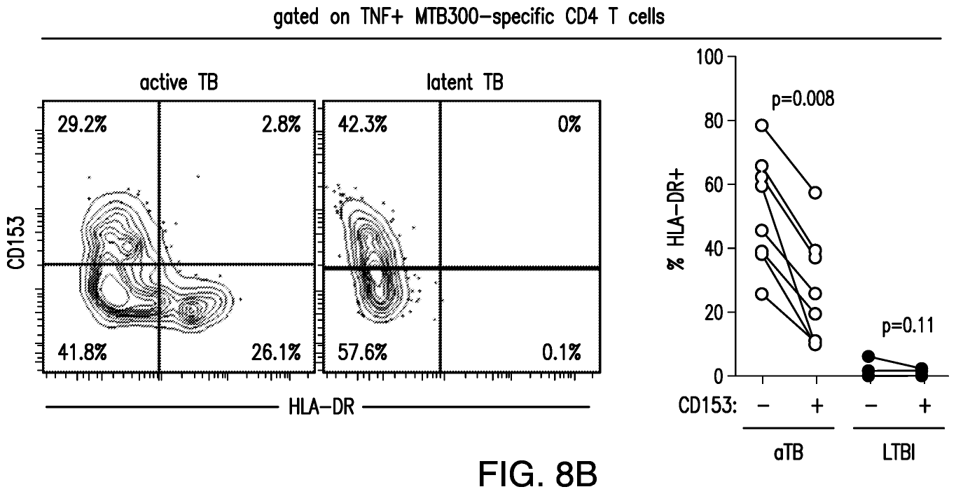

Because CD153 was primarily expressed on less activated KLRG1⁻CD4 T cells in mice, the expression of activation markers KLRG1 and HLA-DR on CD153 expressing Mtb-specific CD4 T cells in humans was examined. During latent Mtb infection, KLRG1 expression was significantly enriched on CD153⁻Mtb-specific T cells (FIG. 8A), and during active TB, CD153⁺ T cells expressed much lower levels of HLA-DR (FIG. 8B). Therefore, similar to what was observed in mice, CD153 expression tends to be associated with less-activated T cells in humans.

CD153 is a major mediator of CD4 T-cell-dependent control of Mtb infection in mice and CD153-expressing CD4 T cells correlate with control of Mtb infection in non-human primates and humans. These data provide a mechanism-based correlate of protection against TB. It was previously shown that CD4 T-cell-derived IFNγ is critical for control of extrapulmonary Mtb infection but has much less of a role in CD4 T-cell-mediated protection in the lungs. Taken together, the data suggests that CD4 T-cell-derived CD153 play a major role in control of pulmonary Mtb infection, whereas CD4 T-cell-derived IFNγ preferentially prevent bacterial dissemination and/or mediate control of infection at extrapulmonary sites.

These data suggest that tracking CD153 induction on Mtb-specific T cells as a potential correlate of protection in the evaluation of vaccine candidates during pre-clinical testing in mice and non-human primates, as well as human TB vaccine trials.

### Example 2

This example demonstrates certain embodiments of the invention.

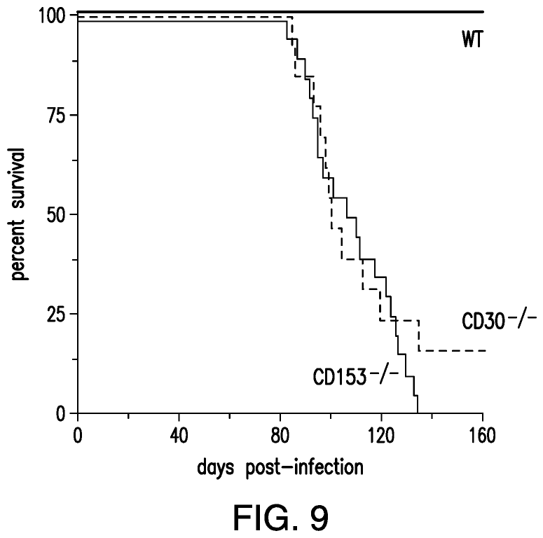

Mice deficient in CD30 (the receptor for CD153) are equally susceptible to Mtb infection as CD153-deficient animals (FIG. 9). This is consistent with the literature that CD153 and CD30 are sole binding partners.

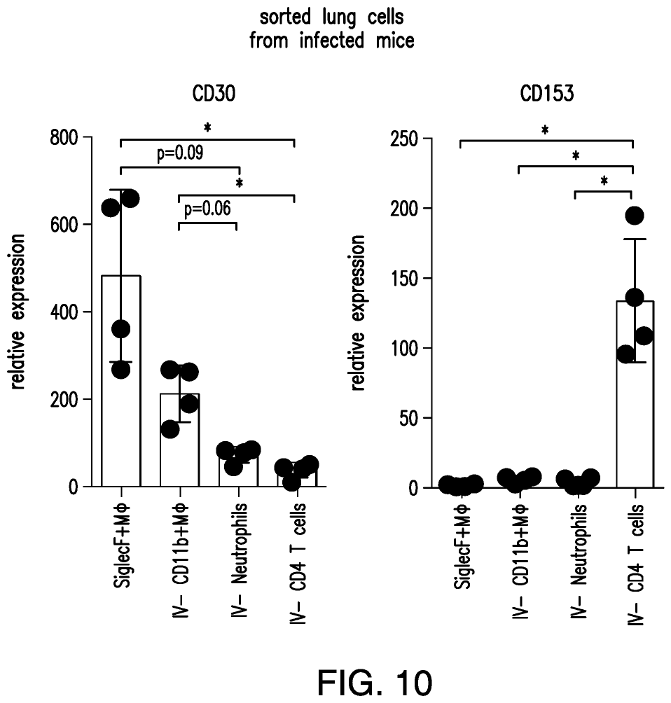

Murine bone marrow-derived macrophages exposed to Mtb in culture rapidly upregulate CD30 to high levels. Moreover, macrophages purified from the lungs of Mtb infected mice express high levels of CD30 (FIG. 10). The data suggest that CD153 on CD4 T cells act by engaging CD30 on Mtb infected macrophages and target CD30 on macrophages to enhance control of Mtb infection.

### Example 3

This example demonstrates certain embodiments of the invention.

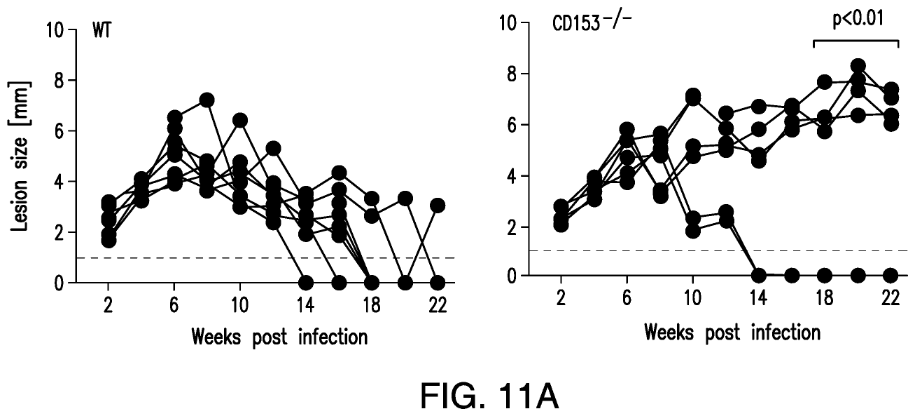

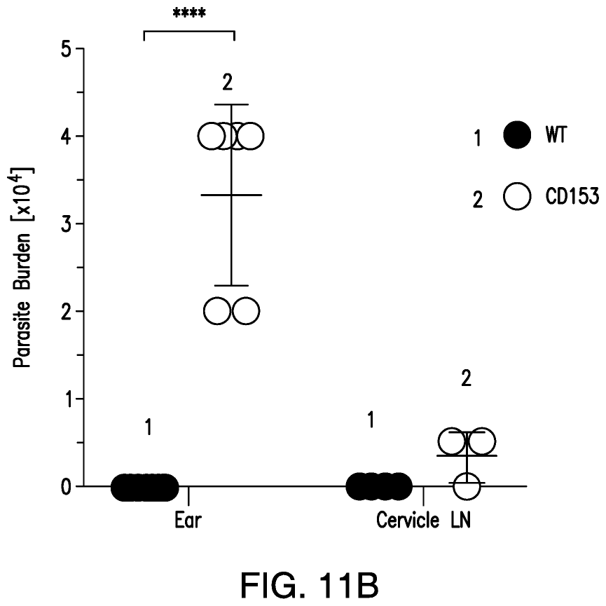

CD153 and CD30 deficient mice are highly susceptible to infection with the intracellular parasite, Leishmania major, suggesting that the CD153/CD30 axis has an important role in control of diverse intracellular pathogens (FIGS. 11A and 11B).

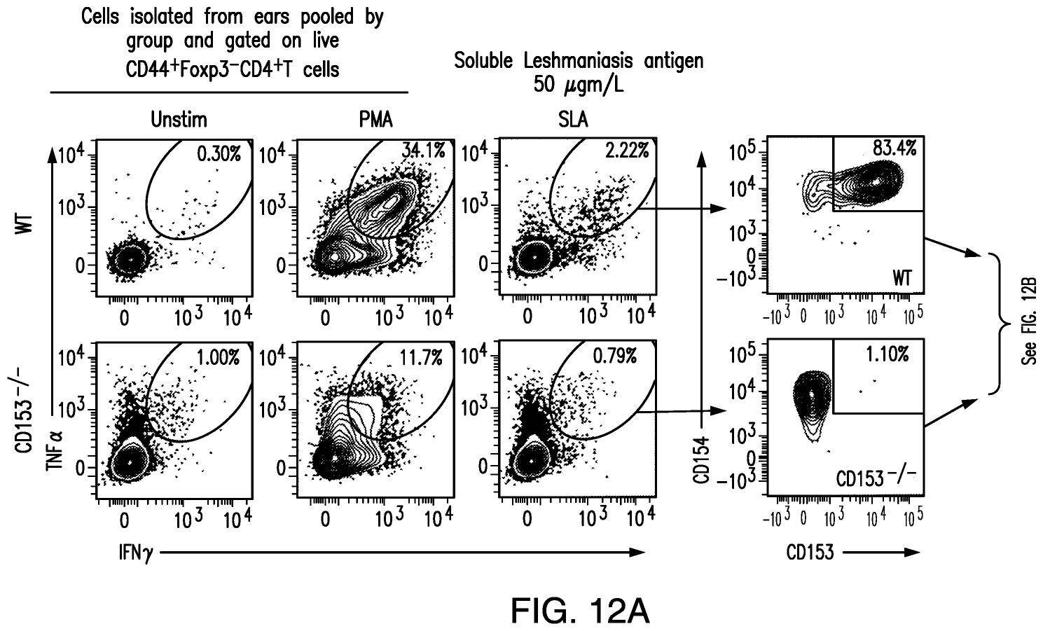

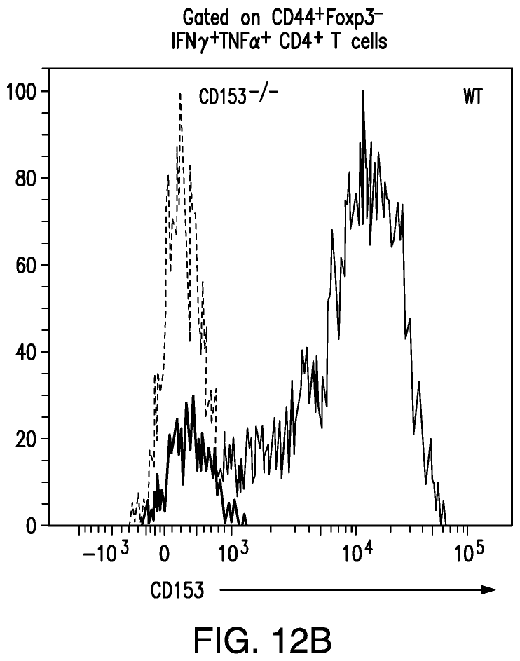

Parasite-specific CD4 T cells isolated from the dermal lesions of L. major infected mice express high levels of CD153, suggesting that monitoring of CD153 expression on parasite specific CD4 T cells as useful for vaccine evaluation (FIGS. 12A and 12B). CD153 is required for control of L. major infection.

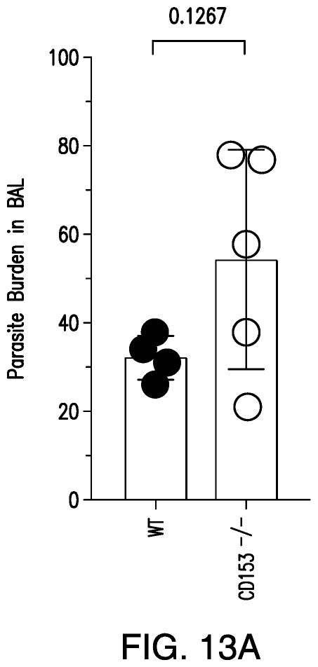

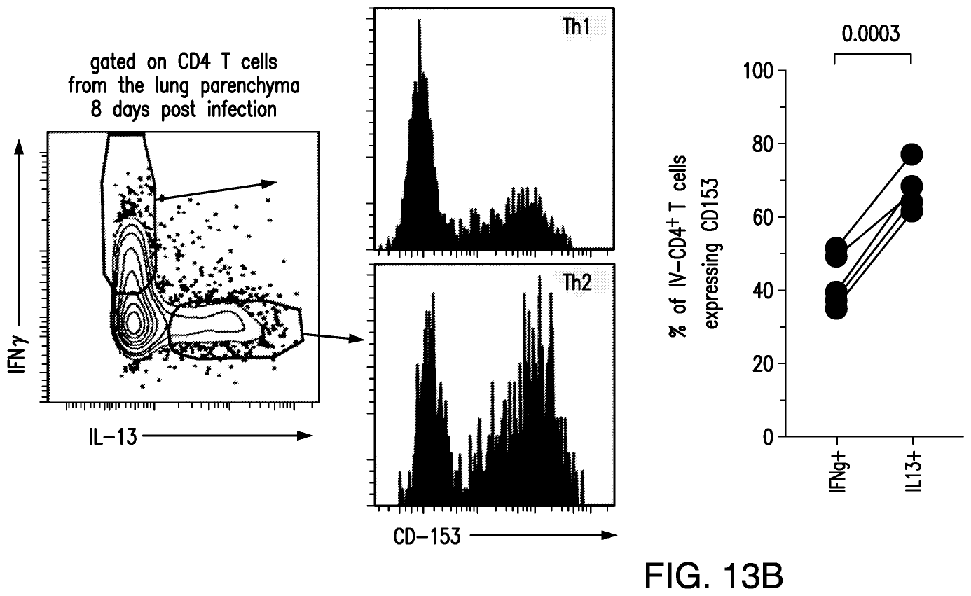

Th2 cells in the lungs of mice experimentally infected with the Ascaris roundworm express very high levels of CD153. There is a trend for CD153-deficient mice to have higher worm burdens in their lungs. Pathogen load differences between WT and CD153 KO mice have not reached statistical significance at early timepoints, although deficient mice are expected to have greater pathogen load over time. See FIGS. 13A and 13B.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of diagnosing infection in a subject, the method comprising:
(a) obtaining a biological sample from the subject;
(b) detecting the expression level of CD153 or CD30 in the sample; and
(c) diagnosing the subject with infection when the expression level of CD153 or CD30 in the sample is detected to be higher compared to the expression level of CD153 or CD30 in a subject without infection.

2.-3. (canceled)

4. The method of claim 1, wherein the expression level of CD153 is detected and the higher CD153 expression level is due to expression in CD4 T cells.

5. The method of claim 1, wherein the biological sample is peripheral blood.

6. The method of claim 1, wherein the biological sample is bronchoalveolar lavage fluid.

7. The method of claim 1, wherein the infection is Mycobacterium tuberculosis infection (Mtb).

8. The method of claim 7, wherein the Mtb infection is a pulmonary Mtb infection.

9. (canceled)

10. The method of claim 1, wherein the infection is a parasitic infection and the parasite is Leishmania major or Ascaris roundworm.

11.-31. (canceled)

32. A method of determining the severity of infection in a subject, the method comprising:
(a) obtaining a biological sample from the subject;
(b) detecting the expression level of CD153 or CD30 in the sample; and
(c) determining the infection to be less severe when the expression level of CD153 or CD30 in the sample is detected to be higher compared to the expression level of CD153 or CD30 in a subject with active infection.

33.-34. (canceled)

35. The method of claim 32, wherein the expression level of CD153 is detected and the higher CD153 expression level is due to expression in CD4 T cells.

36. The method of claim 32, wherein the biological sample is peripheral blood.

37. The method of claim 32, wherein the biological sample is bronchoalveolar lavage fluid.

38. The method of claim 32, wherein the infection is Mycobacterium tuberculosis infection (Mtb).

39. The method of claim 38, wherein the Mtb infection is a pulmonary Mtb infection.

40. (canceled)

41. The method of claim 32, wherein the infection is a parasitic infection and the parasite is Leishmania major or Ascaris roundworm.

42.-48. (canceled)

49. A method of preventing or treating infection in a subject, the method comprising administering to the subject an effective amount of CD4 T cells induced to upregulate CD153.

50. The method according to claim 49, wherein the infection is Mycobacterium tuberculosis infection (Mtb).

51. The method according to claim 50, wherein the Mtb infection is a pulmonary Mtb infection.

52. (canceled)

53. The method according to claim 49, wherein the infection is a parasitic infection and the parasite is Leishmania major or Ascaris roundworm.

54. The method according to claim 49, wherein the CD4 T cells are taken from the subject and administered using adoptive cell transfer.
